(19) United States
(12) Patent Application Publication
(10) Pub. No.: US 2013/0071328 A1
(43) Pub. Date: Mar. 21, 2013

(54) Propynoic Acid Carbamoyl Methyl-Amides and Pharmaceutical Compositions and Methods Based Thereon

(71) Applicants: Nouri NEAMATI, Fullerton, CA (US); Nicos A. PETASIS, Hacienda-Height, CA (US); Roppei YAMADA, Alhambra, CA (US)

(72) Inventors: Nouri NEAMATI, Fullerton, CA (US); Nicos A. PETASIS, Hacienda-Height, CA (US); Roppei YAMADA, Alhambra, CA (US)

(73) Assignee: UNIVERSITY OF SOUTHERN CALIFORNIA, Los Angeles, CA (US)

(21) Appl. No.: 13/427,834

(22) Filed: Mar. 22, 2012

Related U.S. Application Data
(60) Provisional application No. 61/466,407, filed on Mar. 22, 2011.

(51) Int. Cl. C07D 333/24; A61K 49/00; C07D 319/18; C07D 317/60; C07C 235/36; C07D 257/04

(52) U.S. Cl. CPC C07D 333/24; C07C 235/36; C07D 257/04; C07D 319/18; C07D 317/60; A61K 49/00
USPC 424/9.1; 564/158; 549/77; 548/253; 549/362; 549/441; 514/616; 514/438; 514/381; 514/452; 514/466

(57) ABSTRACT

This invention discloses a series of novel propynoic acid carbamoyl methyl-amides (PACMAs), methods for synthesizing the PACMAs and pharmaceutical compositions containing the PACMAs. These novel compounds and compositions show cytotoxicity in cancer cells and are useful as lead compounds for anti-cancer drugs or pharmaceutical agents. This invention also discloses treatment methods that uses the PACMAs and pharmaceutical compositions as well as methods for promoting the release and nuclear localization of the transcription factor Nrf2.

### CROSS-REFERENCE TO RELATED APPLICATIONS

This application claims an invention which was disclosed in Provisional Application No. 61/466,407 filed Mar. 22, 2011, entitled “PROPYONIC ACID CARBAMOYL METHYL-AMIDES AND PHARMACEUTICAL COMPOSITIONS AND METHODS BASED THEREON”. The benefit under 35 USC §119(e) of the United States provisional application is hereby claimed. The above priority application is hereby incorporated herein by reference.

### FIELD OF THE INVENTION

The present invention is directed to the synthesis and use of propynoic acid carbamoyl methyl-amide derivatives. The present invention is also directed to pharmaceutical compositions and treatment methods based on propynoic acid carbamoyl methyl-amides.

### BACKGROUND OF THE INVENTION

Conventional cytotoxic chemotherapies have proven effective in curing and reducing the risk of recurrence for certain types of cancers. However, despite great strikes achieved in the design and discovery of novel anticancer agents, the number of available anticancer agents are still quite limited. It has been estimated that only about one out of every 2,500 cancer lead compound ever made it through to the market. Clearly, the current drug discovery process is not up to the challenge.

Conventional early-phase drug discovery process usually includes lead identification, lead optimization, target identification/validation and preclinical evaluation. The initial focus of lead identification through random screening is to select compounds with desired in vitro biological activities and drug-like properties from a chemically diverse library of small molecules. Evaluation of pharmacokinetics (PK), pharmacodynamics (PD) and metabolism of the active compounds by calculating selective physiochemical properties important for absorption, distribution, metabolism, excretion, and toxicity (ADMET) is also essential to ensure the drug discovery process to be successful and cost-effective. In this random screening approach, it is a major hurdle to identify lead compounds with cancer cell-specific toxicity and selective molecular targets.

Thus, there is still a dire need to develop highly effective and safe anticancer medications.

### BRIEF DESCRIPTION OF THE FIGURES

FIG. 1 shows the prediction of drug adsorption by computational simulation. 3-D Polar surface area in Å² versus partition coefficient (S+logP) calculated by ADMET Predictor.

FIG. 2 shows the cytotoxicity of selected PACMA compounds in a panel of cancer cells. (A) The IC₅₀ values of compounds 1, 2, 4, 6 and 9 in MDA-MB-435 cells range from 0.6 to 6.3 μM. (B) The IC₅₀ values of compound 4 range between 1 and 2.5 μM in tested cell lines using MTT assays.

FIG. 3 shows the colony formation assay of the active PACMA compounds in MDA-MB-435 cells. MDA-MB-435 cells were treated with 1 μM of PACMA compounds. The colony formation observation were consistent with the MTT results. Abbreviations are as follows: PTX-paclitaxel, CDDP-cisplatinum, CONT-vehicle (DMSO) control.

FIG. 4 shows that compound 2 showed in vivo efficacy in an MDA-MB-435 mouse xenograft model. (A) Compound 2 reduced tumor burden. Athymic nude mice implanted with MDA-MB-435 cells were treated with indicated doses of 2 by I. P. administration five times weekly. Values represent the tumor volumes (mean±SEM) for each group. (B) Compound 2 treatment did not cause weight loss. This compound was well tolerated and did not result in any drug-related deaths or changes in body weight.

FIG. 5 shows that cell cycle profiles of MDA-MB-435 cells treated with compound 4. Cells were treated with 5 μM compound 4 for 6, 12, and 24 hours, collected, stained with propidium iodide, and analyzed for perturbation of cell cycle by flow cytometry. Compound 4 induced both S- and G₂/M-arrest.

FIG. 6 shows that compounds 1, 2, and 4 induce apoptosis. Cells were treated with compounds 1, 2 and 4 for 24 and 48 hours, stained with annexin V/propidium iodide and analyzed by flow cytometry. Untreated control cells (48 h) were also included in the analysis. Cells in the bottom left quadrant (annexin V-negative, propidium iodide-negative) are viable, whereas cells in the top left quadrant (annexin V-positive, propidium iodide-negative) are in the early stages of apoptosis, and the cells in the top right quadrant (annexin V-positive, propidium iodide-positive) are in later stages of apoptosis and necrosis.

FIG. 7 shows that p53 and caspase-9 protein expression in MDA-MB-435 cells treated with 1 μM of compound 1. (A) p53 was up-regulated by 1 in a time-dependent manner. (B) The full-length caspase-9 was decreased in response to 1 treatment. Ratios of p53 to GAPDH and full-length caspase-9 to GAPDH were determined using the ImageQuant (GE Healthcare).

FIG. 8 shows that prediction of compound 1-mediated signaling pathways using IPA bioinformatics tool. (A) Ingenuity pathway networks of compound 1-induced cell signaling (score 41) obtained on a set of altered pan-specific proteins. Proteins with their ratio changes and corresponding Swiss-Prot accession numbers were uploaded to IPA software. (-) indicates direct interaction; (---) indicates indirect interaction. Blue bars represent the −log(p-value) of each representative pathway, with the threshold for statistical significance denoted by the thin gray line. Points connected by the solid line represent the ratio of affected molecules versus the total number of molecules in given pathway. (B) Canonical pathway analysis. Fifteen canonical pathways of interested were selected and presented. (C) Quantification of mitochondrial superoxide production upon drug treatment in MDA-MB-435 cells. Cells were treated with compounds 1, 2, 4 and 18 for 24 hours. The untreated and treated cells were incubated with 5 μM MitoSOX for 10 min at 37° C., collected in Hank's balanced salt solution (HBSS), and subjected to flow cytometric analysis. (D) Examination of mitochondrial superoxide production in response to 1, 2, 9 and 18 treatment in MDA-MB-435 cells. Cells were treated with indicated compounds for 24 hours. Untreated and treated cells were incubated with 5 μM of MitoSOX for 10 min at 37° C. and observed under a fluorescence microscope.

FIG. 9 shows the pharmacokinetics distribution of compounds 4 and 9 in mice. Mice were administered 24 mg/kg of compounds 4 and 9 via i.p. administration. Blood samples were collected at 1, 2, 5, and 24 hours for 4 and 0.5, 1, 2, 5, and 24 hours for compound 9. Plasma concentrations of compounds 4 and 9 are plotted against time. The plasma concentration was determined using a validated LCMS method where the lowest level of sensitivity is 1 ng/mL.

### SUMMARY OF THE INVENTION

Disclosed herein is a series of novel propynoic acid carbamoyl methyl-amides with potent cytotoxicity against cancer cells. These compounds interrupted cell cycle progression at low molecular concentrations and induced early and late stage apoptosis.

Accordingly, in one aspect of the present invention, there is provided a series of novel lead compounds useful as potential anticancer agents. Exemplary embodiments in accordance with this aspect of the invention include a propynoic acid carbamoyl methyl amide (PACMA) compound having the general formula:

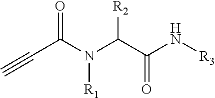

wherein R₁ is hydrogen, alkyl, cycloalkyl, aryl, or heteroaryl; R₂ is hydrogen, alkyl, cycloalkyl, aryl, or heteroaryl; and R₃ is hydrogen, alkyl, aryl, or cycloalkyl.

In other exemplary embodiments of the invention, there is provided a compound having the following general formula:

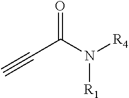

wherein R₁ is hydrogen, alkyl, cycloalkyl, aryl or heteroaryl, R₄ is an alkyl, cycloalkyl, aryl, or heteroaryl.

In another exemplary embodiment of the invention, there is provided a compound having the following general formula:

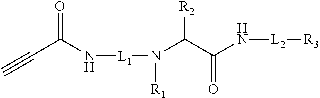

wherein R₁ is hydrogen, alkyl, cycloalkyl, aryl, or heteroaryl; R₂ is hydrogen, alkyl, cycloalkyl, aryl, or heteroaryl; R₃ is hydrogen, alkyl, aryl, or cycloalkyl; L₁ and L₂ are linker groups independently selected from a group consisting of: alkyl, cycloalkyl, aryl, or heteroaryl.

In yet another exemplary embodiment of the invention, there is provided a PACMA compound selected from the group consisting of compounds 1-19 as shown in Scheme 1, compounds 20-35 as shown in Scheme 2, and compounds 36-46 as shown in Scheme 3:

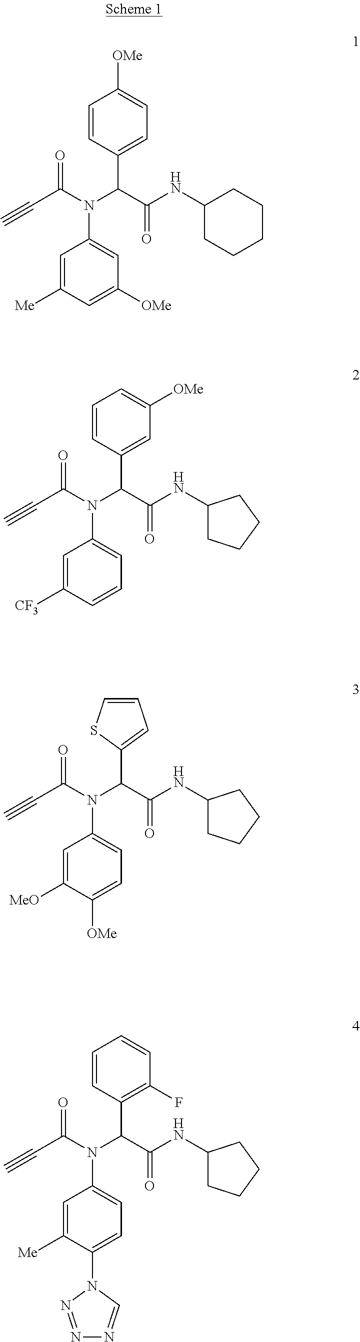 [SMILES: C#CC(=O)N(c1cc(C)cc(OC)c1)C(C(=O)NC1CCCCC1)c1ccc(OC)cc1 ; C#CC(=O)N(c1cccc(C(F)(F)F)c1)C(C(=O)NC1CCCC1)c1cccc(OC)c1 ; C#CC(=O)N(c1ccc(OC)c(OC)c1)C(C(=O)NC1CCCC1)c1cccs1 ; C#CC(=O)N(c1ccc(-n2cnnn2)c(C)c1)C(C(=O)NC1CCCC1)c1ccccc1F]

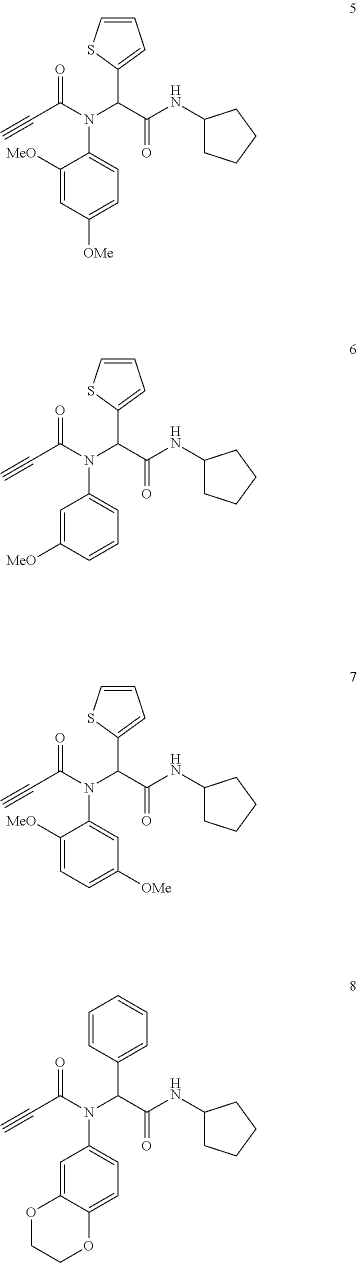 [SMILES: C#CC(=O)N(c1ccc(OC)cc1OC)C(C(=O)NC1CCCC1)c1cccs1 ; C#CC(=O)N(c1cccc(OC)c1)C(C(=O)NC1CCCC1)c1cccs1 ; C#CC(=O)N(c1cc(OC)ccc1OC)C(C(=O)NC1CCCC1)c1cccs1 ; C#CC(=O)N(c1ccc2c(c1)OCCO2)C(C(=O)NC1CCCC1)c1ccccc1]

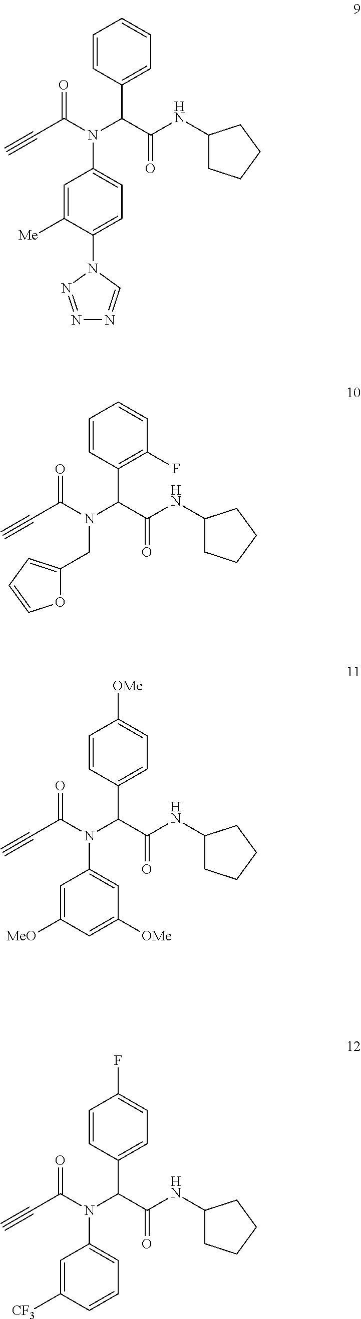 [SMILES: C#CC(=O)N(c1ccc(-n2cnnn2)c(C)c1)C(C(=O)NC1CCCC1)c1ccccc1 ; C#CC(=O)N(Cc1ccco1)C(C(=O)NC1CCCC1)c1ccccc1F ; C#CC(=O)N(c1cc(OC)cc(OC)c1)C(C(=O)NC1CCCC1)c1ccc(OC)cc1 ; C#CC(=O)N(c1cccc(C(F)(F)F)c1)C(C(=O)NC1CCCC1)c1ccc(F)cc1]

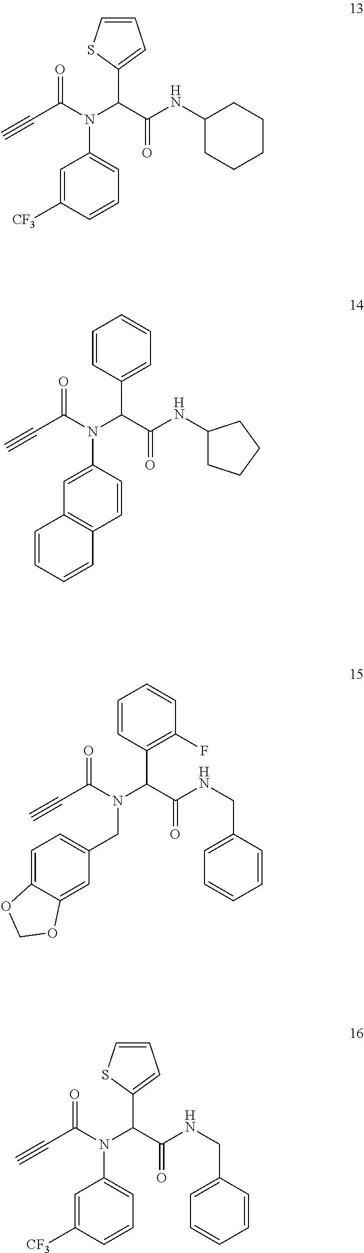 [SMILES: C#CC(=O)N(c1cccc(C(F)(F)F)c1)C(C(=O)NC1CCCCC1)c1cccs1 ; C#CC(=O)N(c1ccc2ccccc2c1)C(C(=O)NC1CCCC1)c1ccccc1 ; C#CC(=O)N(Cc1ccc2c(c1)OCO2)C(C(=O)NCc1ccccc1)c1ccccc1F ; C#CC(=O)N(c1cccc(C(F)(F)F)c1)C(C(=O)NCc1ccccc1)c1cccs1]

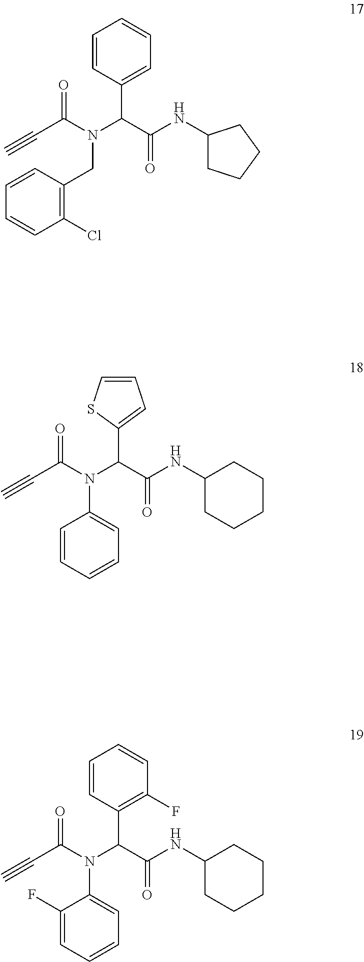 [SMILES: C#CC(=O)N(Cc1ccccc1Cl)C(C(=O)NC1CCCC1)c1ccccc1 ; C#CC(=O)N(c1ccccc1)C(C(=O)NC1CCCCC1)c1cccs1 ; C#CC(=O)N(c1ccccc1F)C(C(=O)NC1CCCCC1)c1ccccc1F]

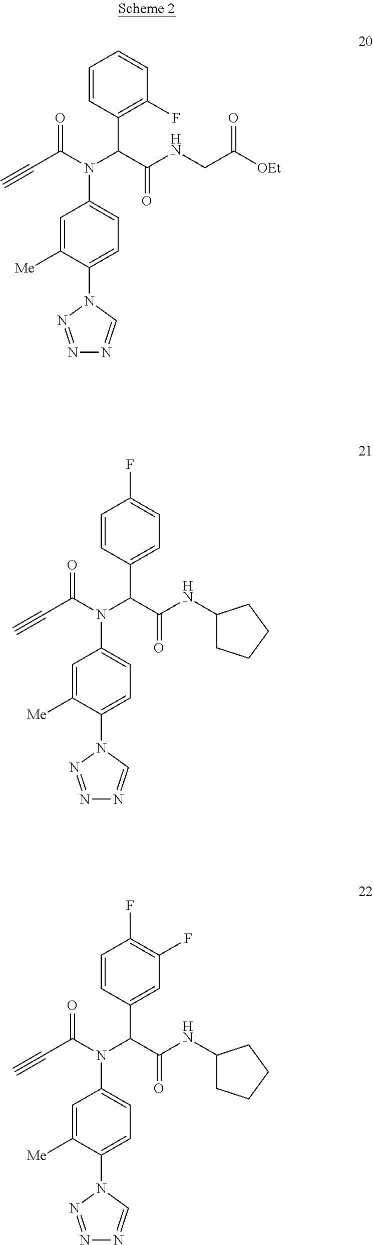 [SMILES: C#CC(=O)N(c1ccc(-n2cnnn2)c(C)c1)C(C(=O)NCC(=O)OCC)c1ccccc1F ; C#CC(=O)N(c1ccc(-n2cnnn2)c(C)c1)C(C(=O)NC1CCCC1)c1ccc(F)cc1 ; C#CC(=O)N(c1ccc(-n2cnnn2)c(C)c1)C(C(=O)NC1CCCC1)c1ccc(F)c(F)c1]

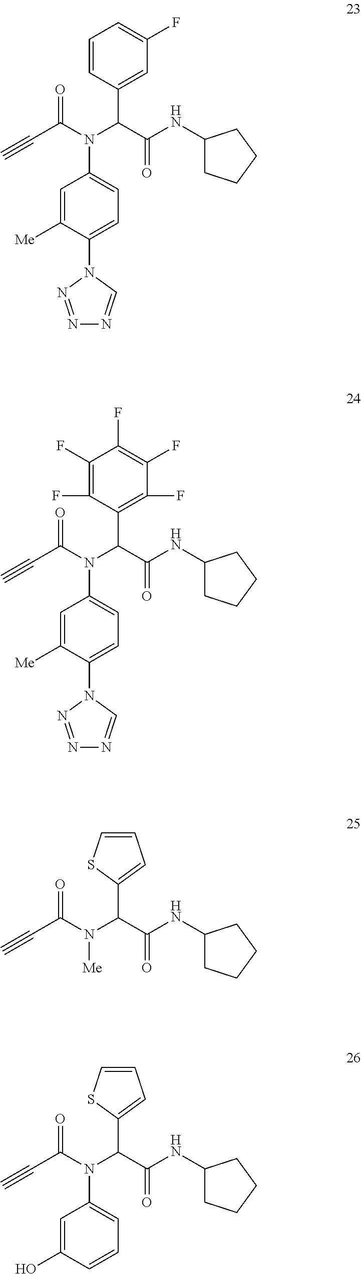 [SMILES: C#CC(=O)N(c1ccc(-n2cnnn2)c(C)c1)C(C(=O)NC1CCCC1)c1cccc(F)c1 ; C#CC(=O)N(c1ccc(-n2cnnn2)c(C)c1)C(C(=O)NC1CCCC1)c1c(F)c(F)c(F)c(F)c1F ; C#CC(=O)N(C)C(C(=O)NC1CCCC1)c1cccs1 ; C#CC(=O)N(c1cccc(O)c1)C(C(=O)NC1CCCC1)c1cccs1]

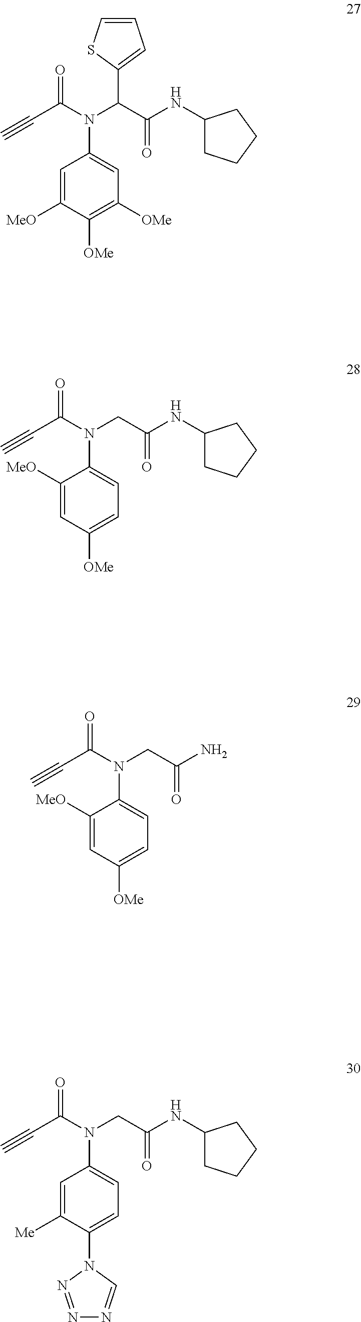 [SMILES: C#CC(=O)N(c1cc(OC)c(OC)c(OC)c1)C(C(=O)NC1CCCC1)c1cccs1 ; C#CC(=O)N(CC(=O)NC1CCCC1)c1ccc(OC)cc1OC ; C#CC(=O)N(CC(N)=O)c1ccc(OC)cc1OC ; C#CC(=O)N(CC(=O)NC1CCCC1)c1ccc(-n2cnnn2)c(C)c1]

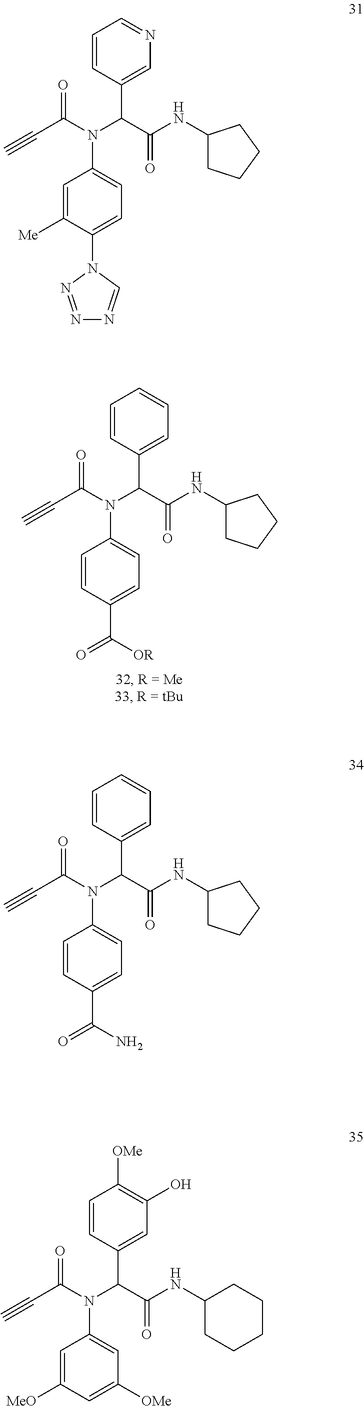

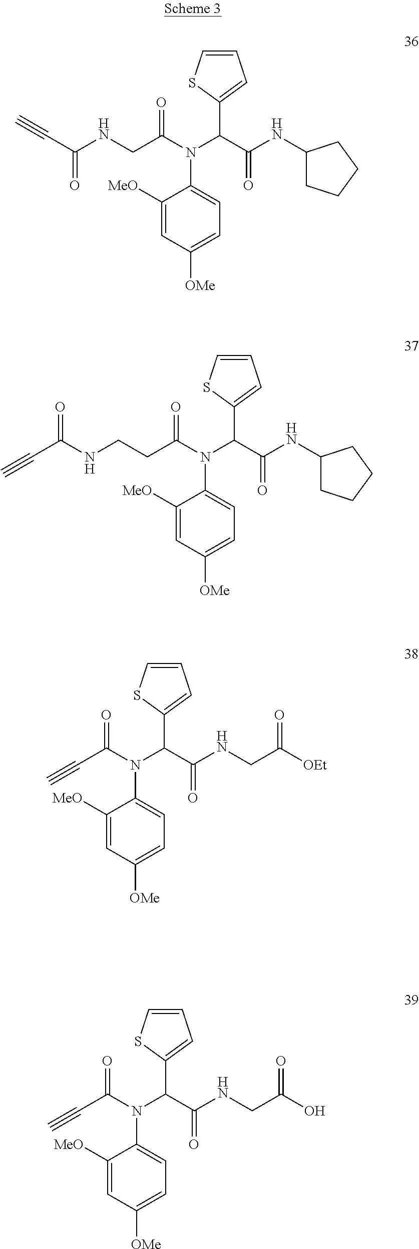 [SMILES: C#CC(=O)NCC(=O)N(c1ccc(OC)cc1OC)C(C(=O)NC1CCCC1)c1cccs1 ; C#CC(=O)NCCC(=O)N(c1ccc(OC)cc1OC)C(C(=O)NC1CCCC1)c1cccs1 ; C#CC(=O)N(c1ccc(OC)cc1OC)C(C(=O)NCC(=O)OCC)c1cccs1 ; C#CC(=O)N(c1ccc(OC)cc1OC)C(C(=O)NCC(=O)O)c1cccs1]

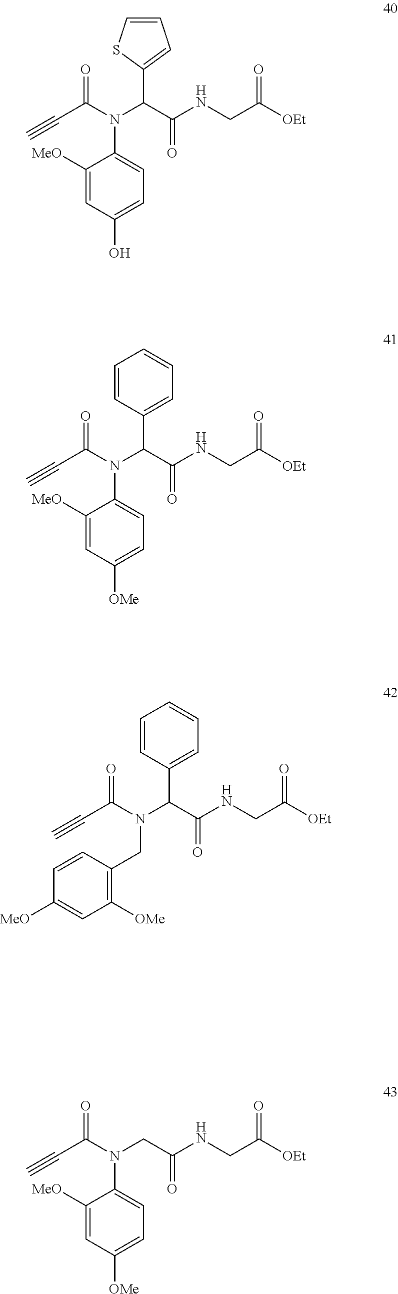 [SMILES: C#CC(=O)N(c1ccc(O)cc1OC)C(C(=O)NCC(=O)OCC)c1cccs1 ; C#CC(=O)N(c1ccc(OC)cc1OC)C(C(=O)NCC(=O)OCC)c1ccccc1 ; C#CC(=O)N(Cc1ccc(OC)cc1OC)C(C(=O)NCC(=O)OCC)c1ccccc1 ; C#CC(=O)N(CC(=O)NCC(=O)OCC)c1ccc(OC)cc1OC]

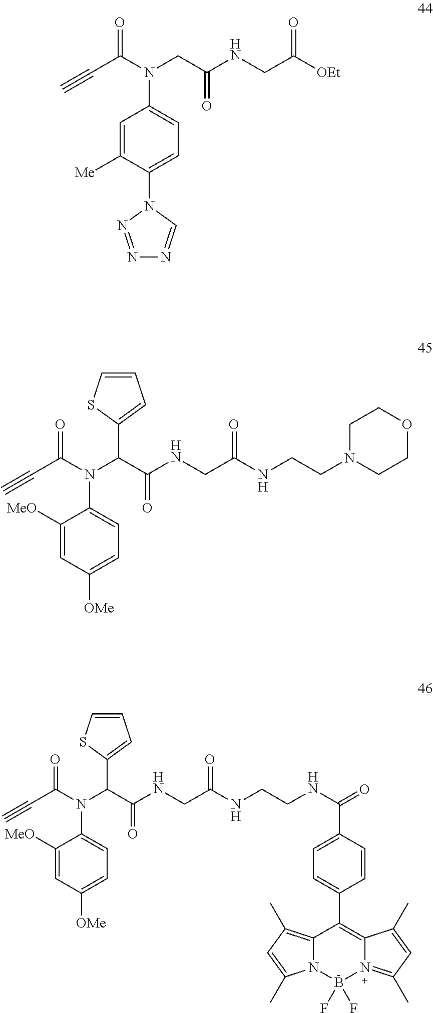

In another aspect, the present invention provides a series of novel compounds useful as diagnostic markers or molecular imaging agents. Exemplary embodiments in accordance with this aspect of the invention include the novel PACMA compounds as described herein above, wherein said compounds contain a substituent that can be used for the imaging of cells, tissues, patient specimens and other preparations, for the purpose of identifying and quantifying associated biomarker proteins for diagnostic applications and biomarker analysis. In other words, PACMAs in accordance with this aspect of the invention are those having the active PACMA functionality as well as a substituent capable of being used as an imaging indicator in a biological or medical diagnostic imagining technique. Exemplary biological or medical diagnostic techniques may include any known techniques in the art, preferably magnetic resonance imaging, and fluorometric analysis of patient specimens or cell cultures. An exemplary embodiment of the aspect of the invention is compound 46, as shown in Scheme 3. When this compound is used in a cell culture it selectively indicates the location of a PACMA target that can be imaged and quantified.

In yet another aspect, the present invention provides a biological or medical diagnostic imagining technique utilizing the novel PACMA compounds as described above. Embodiments in accordance with this aspect of the invention generally include the steps of administering to a subject a PACMA compound having a substituent capable of being detected by the imaging technique; and detecting the PACMA compound using the imaging technique. The resulting data may be further processed according to the needs of a diagnosis or analysis. Exemplary imagining techniques are as described above.

In yet another aspect, the present invention also provides a pharmaceutical composition useful for treating cancer. Exemplary embodiments in accordance with this aspect of the invention are pharmaceutical compositions that include any of the compounds as described above and a pharmaceutically acceptable carrier.

In still another aspect, the present invention provided a treatment method for treating cancer. Exemplary embodiments in accordance with this aspect of the invention generally comprises the step of administering to a person in need thereof an effective amount of the pharmaceutical composition as described above.

In still a further aspect, the present invention provides a method for promoting the release and nuclear localization of the transcription factor Nrf2. Exemplary embodiments in accordance with this aspect of the invention generally include the step of administering an effective amount of the pharmaceutical compositions as described above.

Other aspects and advantages of the present invention will become apparent from the following detailed description and the appended claims.

### DETAILED DESCRIPTION

### Abbreviations.

ADMET=Absorption, Distribution, Metabolism, Excretion, and Toxicity
DCFH=2,7-dichlorodihydrofluorescein
DMSO=dimethyl sulfoxide.
HBA=hydrogen-bond acceptor
HBD=hydrogen-bond donor
HSP=heat shock protein
HTS=high-throughput screening
I. P.=intraperitoneal
IPA=Ingenuity pathway analysis
MMA=monomethylarsonous acid
MW=molecular weight
MlogP=Moriguchi octanol-water partition coefficient
Nrf2=nuclear factor erythroid 2-related factor 2
PACMAs=propynoic acid carbamoyl methyl-amides
PBS=phosphate buffered-saline
PK=pharmacokinetics
PD=pharmacodynamics
PSA=three-dimensional polar surface area
ROS=reactive oxygen species
SAR=structure activity relationship
SEM=standard error of mean
SOD1=superoxide dismutase
S+LogP=SimulationsPlus, Inc., model of log P

### DEFINITIONS

As used herein, the nomenclature alkyl, alkoxy, carbonyl, etc. is used as is generally understood by those of skill in this art.

As used in this specification, alkyl groups can include straight-chained, branched and cyclic alkyl radicals containing up to about 20 carbons, or 1 to 16 carbons, and are straight or branched. Exemplary alkyl groups herein include, but are not limited to, methyl, ethyl, propyl, isopropyl, isobutyl, n-butyl, sec-butyl, tert-butyl, isopentyl, neopentyl, tert-pentyl, and isohexyl. As used herein, lower alkyl refer to carbon chains having from about 1 or about 2 carbons up to about 6 carbons. Suitable alkyl groups may be saturated or unsaturated. Further, an alkyl may also be substituted one or more times on one or more carbons with substituents selected from a group consisting of C1-C15 alkyl, allyl, allenyl, alkenyl, C3-C10 carbocycle, C3-C7 heterocycle, aryl, fluoro, chloro, bromo, iodo, hydroxy, amino, cyano, oxo, thio, alkoxy, formyl, carboxy, carboxamido, phosphoryl, phosphonate, phosphonamido, sulfonyl, alkylsulfonate, arylsulfonate, and sulfonamide. Additionally, an alkyl group may contain up to 10 heteroatoms, in certain embodiments, 1, 2, 3, 4, 5, 6, 7, 8 or 9 heteroatom substituents. Suitable heteroatoms include nitrogen, oxygen, sulfur, and phosphorous. As used herein, alkenyl and alkynyl carbon chains, if not specified, contain from 2 to 20 carbons, or 2 to 16 carbons, and are straight or branched. Alkenyl carbon chains of from 2 to 20 carbons, in certain embodiments, contain 1 to 8 double bonds, and the alkenyl carbon chains of 2 to 16 carbons, in certain embodiments, contain 1 to 5 double bonds. Alkynyl carbon chains of from 2 to 20 carbons, in certain embodiments, contain 1 to 8 triple bonds, and the alkynyl carbon chains of 2 to 16 carbons, in certain embodiments, contain 1 to 5 triple bonds. As used herein, “alkoxy” refers to RO—, in which R is alkyl, including lower alkyl. As used herein, “aryloxy” refers to RO—, in which R is aryl, including lower aryl, such as phenyl.

As used herein, “cycloalkyl” refers to a mono- or multicyclic ring system, in certain embodiments of 3 to 10 carbon atoms, in other embodiments of 3 to 6 carbon atoms. The ring systems of the cycloalkyl group may be composed of one ring or two or more rings which may be joined together in a fused, bridged or spiro-connected fashion. As used herein, “aryl” refers to aromatic monocyclic or multicyclic groups containing from 3 to 16 carbon atoms.

As used in this specification, aryl groups are aryl radicals which may contain up to 10 heteroatoms, in certain embodiments, 1, 2, 3, or 4 heteroatoms. An aryl group may also be optionally substituted one or more times, in certain embodiments, 1 to 3 or 4 times with an aryl group or a lower alkyl group and it may be also fused to other aryl or cycloalkyl rings. Suitable aryl groups include, for example, phenyl, naphthyl, tolyl, imidazolyl, pyridyl, pyrroyl, thienyl, pyrimidyl, thiazolyl, and furyl groups. As used in this specification, a ring is defined as having up to 20 atoms that may include one or more nitrogen, oxygen, sulfur, or phosphorous atoms, provided that the ring can have one or more substituents selected from the group consisting of hydrogen, alkyl, allyl, alkenyl, alkynyl, aryl, heteroaryl, chloro, iodo, bromo, fluoro, hydroxy, alkoxy, aryloxy, carboxy, amino, alkylamino, dialkylamino, acylamino, carboxamido, cyano, oxo, thio, alkylthio, arylthio, acylthio, alkylsulfonate, arylsulfonate, phosphoryl, phosphonate, phosphonamido, and sulfonyl, and further provided that the ring may also contain one or more fused rings, including carbocyclic, heterocyclic, aryl or heteroaryl rings.

As used herein, “heteroaryl” refers to a monocyclic or multicyclic aromatic ring system, in certain embodiments, of about 5 to about 15 members where one or more, in one embodiment 1 to 3, of the atoms in the ring system is a heteroatom, that is, an element other than carbon, including but not limited to, nitrogen, oxygen or sulfur. The heteroaryl group may be optionally fused to a benzene ring. Heteroaryl groups include, but are not limited to, furyl, imidazolyl, pyrrolidinyl, pyrimidinyl, tetrazolyl, thienyl, pyridyl, pyrrolyl, N-methylpyrrolyl, quinolinyl, and isoquinolinyl. As used herein, “heterocyclyl” refers to a monocyclic or multicyclic non-aromatic ring system, in one embodiment of 3 to 10 members, in another embodiment of 4 to 7 members, in a further embodiment of 5 to 6 members, where one or more, in certain embodiments, 1 to 3, of the atoms in the ring system is a heteroatom, that is, an element other than carbon, including but not limited to, nitrogen, oxygen, or sulfur. In embodiments where the heteroatom(s) is(are) nitrogen, the nitrogen is optionally substituted with alkyl, alkenyl, alkynyl, aryl, heteroaryl, aralkyl, heteroaralkyl, cycloalkyl, heterocyclyl, cycloalkylalkyl, heterocyclylalkyl, acyl, guanidino, or the nitrogen may be quaternized to form an ammonium group where the substituents are selected as above.

### General Methods for the Synthesis of PACMSs

The propynoic acid carbamoyl methyl-amide compounds disclosed herein may be conveniently prepared by several methods known in the art, including the use of a single step synthesis by employing the four-component Ugi reaction as shown in Method 1 below:

Method 1. Synthesis of PACMAs (E) using Ugi Reaction

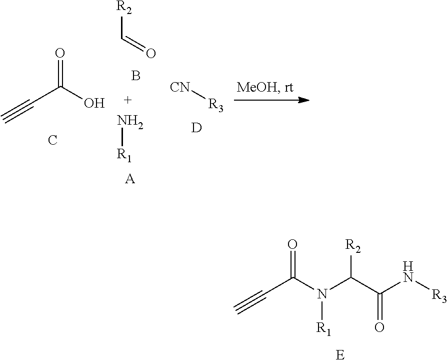

In a second exemplary method, PACMAs of the present invention may be synthesized by an amide formation method as shown in Method 2 below:

### Method 2. PACMAs (E) Synthesis Through Amide Formation

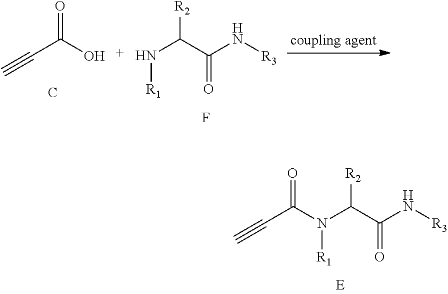

In a third exemplary method, PACMAs of the present invention may be synthesized by Method 3 as shown below:

### Method 3. PACMAs (E) Synthesis Through Amide Formation

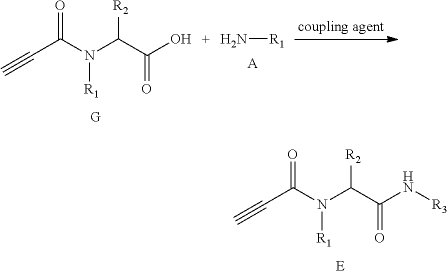

In a fourth exemplary method, PACMAs of the present invention may be synthesized by Method 4 as shown below:

### Method 4. PACMAs Synthesis Through Amide Formation

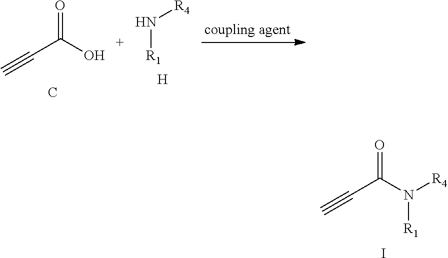

The PACMAs according to the present invention have the general formula (E), as shown in Methods 1-3 above, wherein R₁ is hydrogen, alkyl, aryl, or heteroaryl; R₂ is hydrogen, alkyl, cycloalkyl, aryl, or heteroaryl; and R₃ is alkyl, aryl, heteroaryl, benzyl or cycloalkyl, or the general formula (I) as shown in method 4, wherein R₁ is hydrogen, alkyl, cycloalkyl, aryl or heteroaryl; and R₄ is alkyl, cycloalkyl, aryl, or heteroaryl.

The term “aryl” as used herein means monocyclic or condensed ring aromatic hydrocarbons. Examples of the aryl are phenyl, naphthyl, and the like.

The term “heteroaryl” as used herein means a 5 to 6 membered aromatic heterocyclic group which contains one or more hetero atoms selected from the group consisting of nitrogen, oxygen, and sulfur atoms in the ring and may be fused with a carbocyclic ring or other heterocyclic ring at any possible position.

The term “cycloalkyl” as used herein refers to a cyclized alkyl exemplified by cyclopropyl, cyclobutyl, cyclopentyl, cyclohexyl, cycloheptyl, and the like.

Compounds in accordance with embodiments of the present invention may contain one or more chiral centers and/or double bonds and, therefore, may exist as stereoisomers such as double-bond isomers (i.e. geometric isomers), enantiomers, or diastereomers. According to the invention, the chemical structures depicted herein, and therefore the compounds of the invention encompass all of the corresponding compounds' enantiomers and stereoisomers, that is, both the stereomerically pure form (e.g. geometrically pure, enantiomerically pure, or diastereomerically pure) and enantiomeric and stereoisomeric mixtures.

Enantiomeric and diastereomeric mixtures can be resolved into their component enantiomers or stereoisomers by well-known methods, such as chiral-phase gas chromatography, chiral-phase high performance liquid chromatography, crystallizing the compound as a chiral salt complex, or crystallizing the compound in a chiral solvent. Enantiomers and diastereomers can also be obtained from diastereomerically- or enantiomerically-pure intermediates, reagents, and catalysts by well-known asymmetric synthetic methods.

Each of the corresponding PACMA compounds 1-46 (Schemes 1-3, above), were obtained in good yields by reacting equimolar amounts of a primary amine or aniline component A, an aryl or heteroaryl aldehyde B, propynoic acid C, and a cycloalkyl or benzyl isocyanide D in methanol at room temperature over 48 hours, followed by purification via chromatography and recrystallization.

It should be understood that the examples of Schemes 1-3 are provided in order to demonstrate and further illustrate certain embodiments and aspects of the present invention and are not to be construed as limiting the scope thereof.

### Pharmaceutical Compositions

The propynoic acid carbamoyl methyl-amide compounds of the present invention can be formulated as pharmaceutical compositions by combining the selected PACMA with one or more pharmaceutically acceptable carriers.

“Carriers” as used herein include pharmaceutically acceptable carriers, excipients, or stabilizers which are nontoxic to the cell or mammal being exposed thereto at the dosages and concentrations employed. Often the physiologically acceptable carrier is an aqueous pH buffered solution. Examples of physiologically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants.

The route of administration is in accord with known methods, e.g. injection or infusion by intravenous, intraperitoneal, intracerebral, intramuscular, intraocular, intraarterial or intralesional routes, topical administration, or by sustained release systems. Dosages and desired drug concentrations of pharmaceutical compositions of the present invention may vary depending on the particular use envisioned. The determination of the appropriate dosage or route of administration is well within the skill of an ordinary physician. Animal experiments provide reliable guidance for the determination of effective doses for human therapy. Interspecies scaling of effective doses can be performed following the principles laid down by Mordenti, J. and Chappell, W. “The use of interspecies scaling in toxicokinetics” In Toxicokinetics and New Drug Development, Yacobi et al., Eds., Pergamon Press, New York 1989, pp. 42-96.

### Drug-Like Properties of the PACMA Compounds

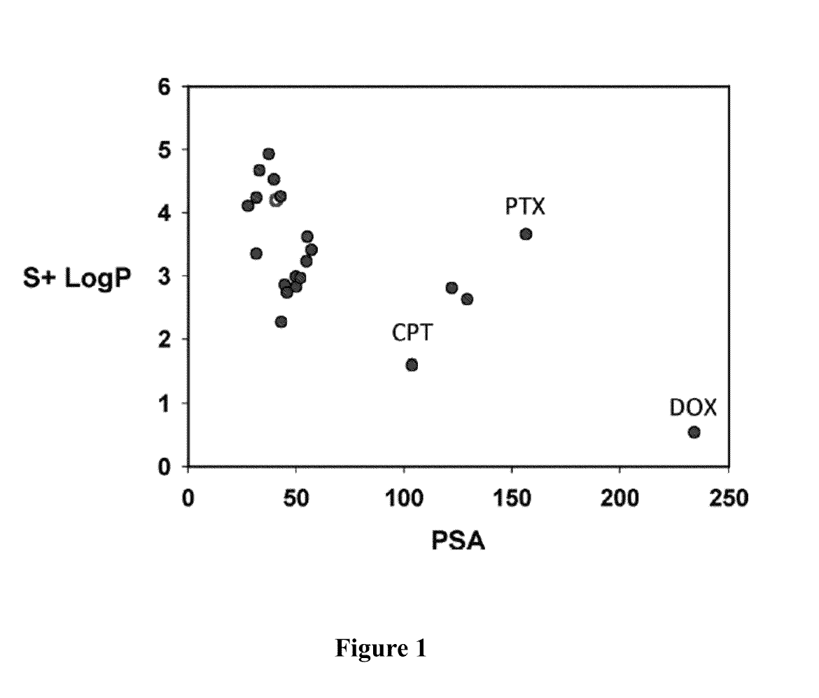

Oral bioavailability is a desirable property of investigational compounds in the drug discovery process. Lipinski's rule-of-five is a simplified model to predict the absorption and intestinal permeability of a compound. In this model, compounds are considered likely to be well absorbed when they possess c10gP <5, molecular weight <500, number of H-bond donors <5, and number H-bond acceptors <10. Earlier studies by Palm et al. and Kelder et al. recommend that compounds with polar surface area >140 Å² will likely have poor absorption (<10%), whereas compounds with polar surface area <60 Å² are predicted to show complete absorption (>90%). The calculated atom-based Log-P (S+logP) values of the PACMAs ranges from 2.2 to 4.4, and H-bond donor and acceptor counts are <5 and <10, respectively. As shown in FIG. 1 and Table I, all active PACMA compounds fall within the desirable range favoring excellent oral bioavailability, whereas the reference compounds doxorubicin and paclitaxel have maximum polar surface areas of >150 Å² and camptothecin has a moderate polar surface area of 103 Å². In conclusion, all PACMA compounds demonstrate desirable physicochemical properties and therefore, are potentially promising for further optimization and experimental evaluations.

### PACMAs Exhibit Cytotoxicity at Low Micromolar Concentrations in a Panel of Human Cancer Cell Lines

The 19 PACMA compounds were tested in eight human cancer cell lines derived from different tumor origins, including the MDA-MB-435 breast cancer, HCT116 p53^{+/+}, HCT116 p53^{-/-}, and HT29 colon cancer, HEY and doxorubicin-resistant NCI/ADR-RES ovarian cancer, and UMUC3 and 5637 bladder cancer cell lines (Table 2). Remarkably, all compounds showed potent cytotoxicity in the NCI/ADR-RES cell line, and in fact, this cell line exhibited the greatest sensitivity to these compounds (Table 2). NCI/ADR-RES cells have been shown to overexpress multidrug resistance transporters and p-glycoproteins, which represents the most common mechanisms for acquisition of resistance by cancer cells.

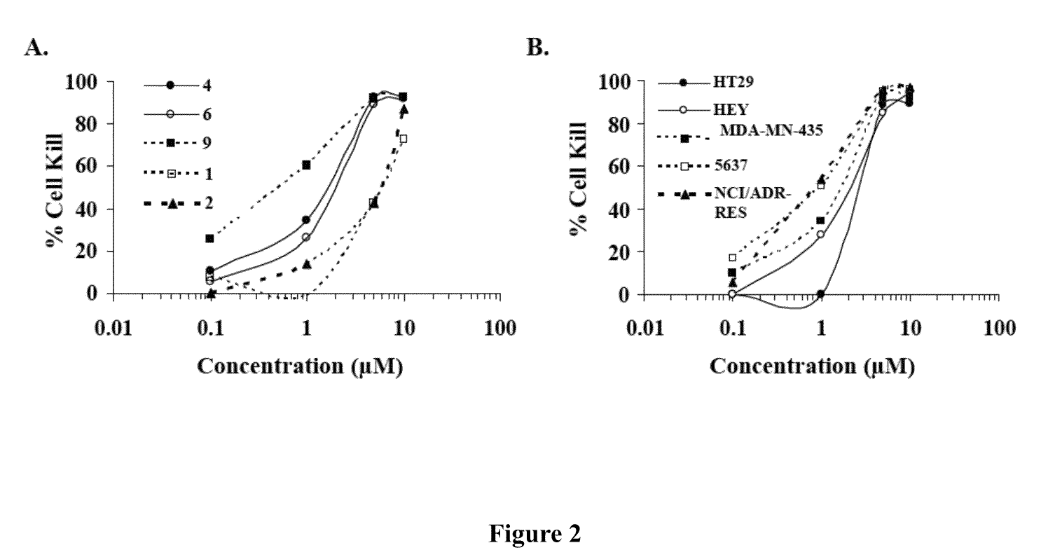

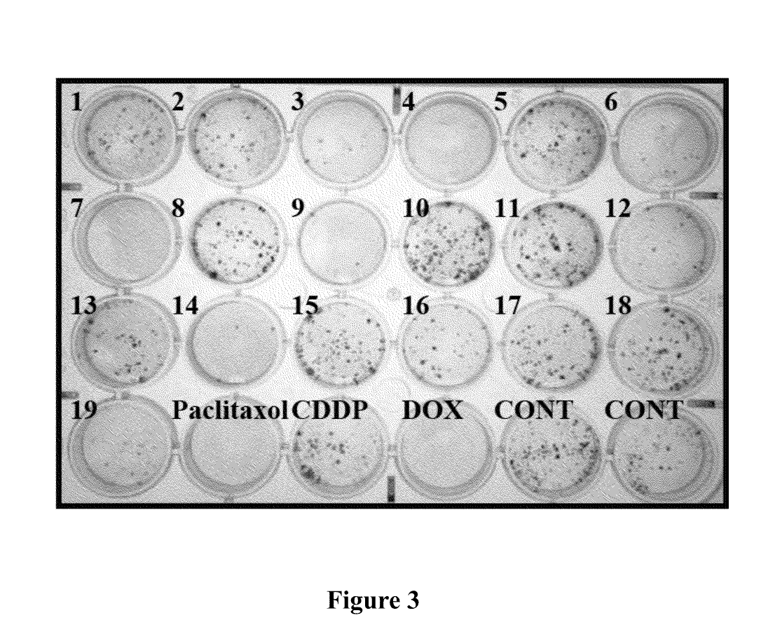

The potent cytotoxicity of these compounds in NCI/ADR-RES cells indicates their promising therapeutic potential for treatment of human cancers that have developed resistance to standard chemotherapeutic agents, such as doxorubicin and paclitaxel. In addition to NCI/ADR-RES cells, Rb-null 5637 cells were also sensitive to these compounds, whereas Rb-expressing UMUC3 cells were the least sensitive among the cell lines tested (Table 2). When tested in MDA-MB-435 cells, compound 9 appeared to be the most potent analogue (FIG. 2A). Among the selected cell lines, compound 4 showed remarkable potency in 5637 cells (FIG. 2B). It has been reported that 5637 bladder cancer cells lack a functional Rb protein and are highly metastatic. On the basis of observed Rb-dependency by these compounds, they are predicted to have potential applications in highly malignant bladder cancers. To further confirm the MTT observations, we performed colony formation assays on all compounds at 1 μM in MDA-MB-435 cells (FIG. 3). The results were conclusive and consistent with the MTT observations. In addition to these 19 compounds, we also tested 61 compounds without the propynamide functional group in MDAMB-435 cells, of which 13 compounds exhibited moderate cytotoxicity around 40% at 10 μM (data not shown). However, when tested in NCI/ADR-RES cells, they failed to inhibit colony formation. This shows that the propynamide moiety is critical in inducing cytotoxicity for this class of compounds.

### Compound 2 Exhibits Promising In Vivo Efficacy in an MDA-MB-435 Mouse Xenograft Model

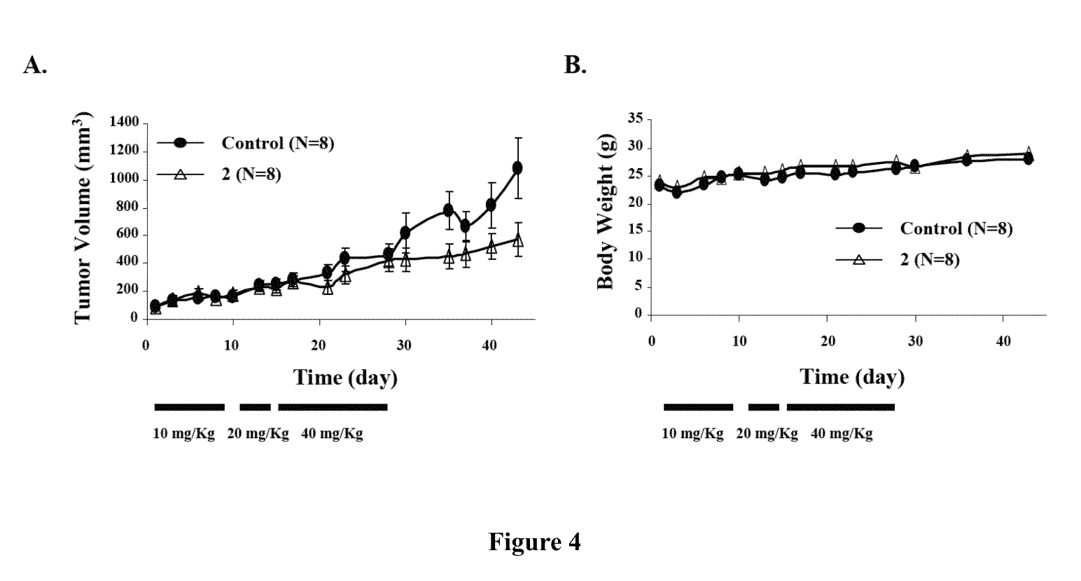

To investigate the in vivo efficacy of these compounds, we selected compound 2 and evaluated its anticancer activity in a nude mouse xenograft model using MDA-MB-435 cells. Animals were treated with daily intraperitoneal (i.p.) injections of DMSO in sesame oil (vehicle control) or compound 2 at 10 mg/kg for 9 days, followed by 20 mg/kg for 5 days and concluding with 40 mg/kg for 12 days. After 26 days of dosing, the drug treatment was discontinued. Mice were monitored daily for an additional 17 days, and tumors were measured twice per week. FIG. 4A summarizes the tumor volume (mean±SEM) of the treatment groups throughout the observation time. Compound 2 significantly reduced tumor burden in this xenograft model.

The possible toxicity elicited by compound 2 was also evaluated by following the body weight of the mice over the course of treatment and histological examination of the organs. The treatment was well-tolerated and did not result in any drug-related death or body weight loss (FIG. 4B). Specifically, the untreated control mice had an average weight of 23±2 g (mean±SD) before the experiment and 28±2 g after the experiment. Mice treated with compound 2 had a comparable average weight of 29±2 g after treatment.

Furthermore, histopathological examinations of the organs derived from at least three mice in the treatment group showed no histological evidence of organ toxicity (data not shown).

### Compound 4 Induces S and G₂/M Cell Cycle Arrest in MDA-MB-435 Cells

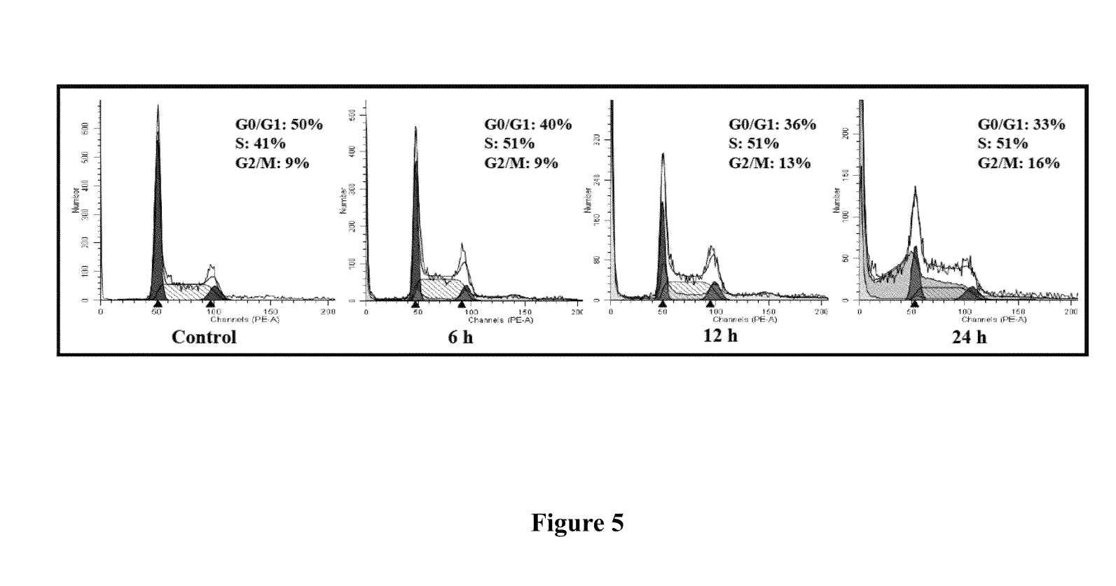

Compound 4 is one of the most active compounds in the MDA-MB-435 cell line (Table 2). Hence, we selected this compound for further examination to determine its ability to induce cell cycle perturbation in MDA-MB-435 cells. Cells were treated with compound 4 for 6, 12, and 24 hours. The analysis of DNA profiles by flow cytometry demonstrated that compound 4 was able to block cell cycle progression at both S and G₂/M phases (FIG. 5). In particular, at 24 hours, there was a 10% and 7% increase in the number of cells retained in S and G₂/M phases, respectively, when compared with untreated control cells.

The ability of compound 4 to disrupt cell cycle progression makes it an interesting agent for combination treatments with drugs acting at different stages of cell cycle. For example, compound 4 may be used in combination with other agents that have specific activity in cells in their S or G₂/M phases. Some exemplary combination may include a chemotherapy agent currently used in standard therapy, including, but not limited to: taxol, doxorubicin, kinase inhibitors, and therapeutic antibodies.

### Compounds 1, 2 and 4 Triggers Apoptosis

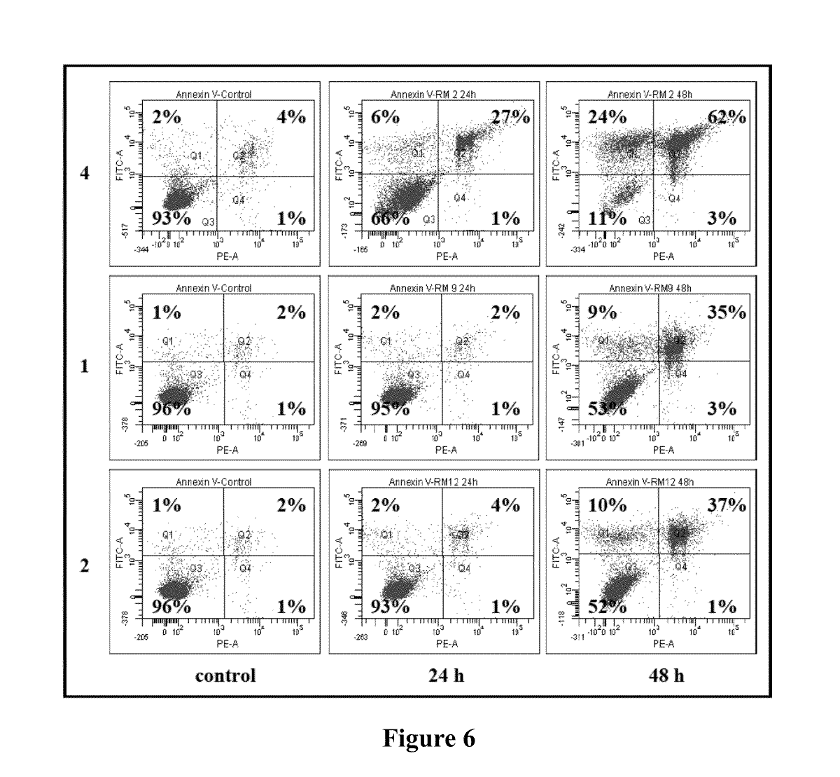

Most cytotoxic anticancer drugs induce apoptosis. To understand the mechanisms underlying the cytotoxicity of PACMAs, we measured the percentage of early and late apoptotic cells in MDA-MB-435 cells treated with compounds 1, 2, and 4, respectively, by flow cytometry. An early event in apoptotic cell death is the translocation of phosphatidyl-serine residues to the outer cell membrane. This event precedes nuclear breakdown, DNA fragmentation, and appearance of most apoptosis-associated molecules and is readily distinguished from the late apoptotic processes by annexin V/propidium iodide binding assay. As shown in FIG. 6, the percentage of early apoptotic cells reached 24% after 48 hours exposure to compound 4 and the ratio of late apoptotic cells was significantly increased to 27% and 62% after 24 and 48 hours treatment, respectively. The original lead compounds 1 and 2, failed to induce early apoptosis at 24 hours, and the early apoptotic cells were slightly increased by 8% after 48 h treatment. However, the percentage of late apoptotic cells reached 35% and 37% for 1 and 2, respectively, after 48 hours treatment, suggesting a less marked and delayed induction of apoptosis by these two compounds. Taken together, these observations implied that apoptosis might be one of the mechanisms utilized by these compounds to elicit cytotoxicity. It is known that apoptosis signaling pathways have a profound effect on both cancer progression and response to chemotherapy.

### Compound 1 Targets Pro-Apoptotic p53 and Caspase-9

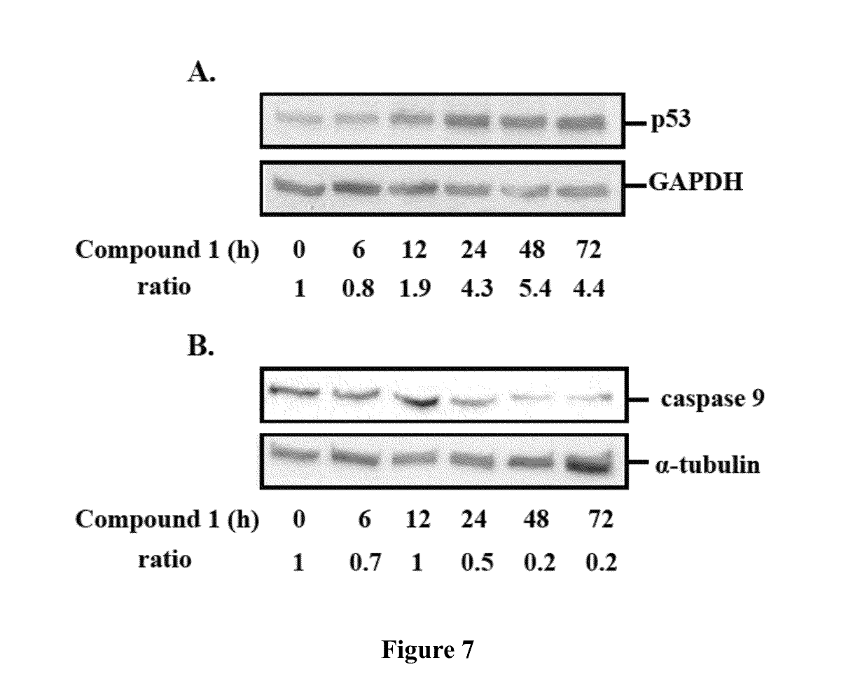

Among the proteins associated with apoptosis signaling, p53 and caspase-9 are the most commonly activated pro-apoptotic proteins by apoptosis-inducing agents. Thus, as a preliminary mechanistic investigation, we examined the cellular effect of the original lead compound 1 on apoptotic signaling through the protein expression changes of p53 and caspase-9. As shown in FIG. 7A, the protein level of p53 was up-regulated by compound 1 after 24 hours treatment and the activation of p53 was sustained for 72 hours (lanes 4-6 versus lane 1), suggesting the involvement of p53 in compound 1-mediated cytotoxicity. Several lines of evidence have demonstrated the activation of caspases during p53-mediated apoptosis in both cell-based and cell-free systems. We, therefore, measured the protein levels of full-length caspase-9 in response to compound 1 treatment by Western blotting. A 2-fold decrease in the full-length caspase-9 protein level was observed after 24 hours, and the change was maintained up to 72 hours (FIG. 7B). These observations led us to hypothesize that the decrease in full-length caspase-9 protein level is due to its cleavage and activation in response to apoptotic signaling initiated by compound 1. This suggests that compound 1 elicits cytotoxicity through activating p53 as well as the downstream signaling cascades of p53-mediated apoptotic events.

### Signaling Network Analysis Using the Ingenuity Pathway Analysis (IPA) Platform

To better understand the mechanisms of action of this class of novel PACMA compounds, we subjected cell lysates from untreated and compound 1-treated MDA-MB-435 cells to the Kinexus 628-antibody microarray analysis in search of potential signaling molecules; 350 pan-specific and 270 phospho-site antibodies were included to track changes in protein expression and phosphorylation status. The cell lysates were run in parallel on the Kinexus antibody chip and the differential binding of dye-labeled proteins was detected and quantified. Proteins with significant expression changes were then identified and used as the basis for signaling pathway interpretation.

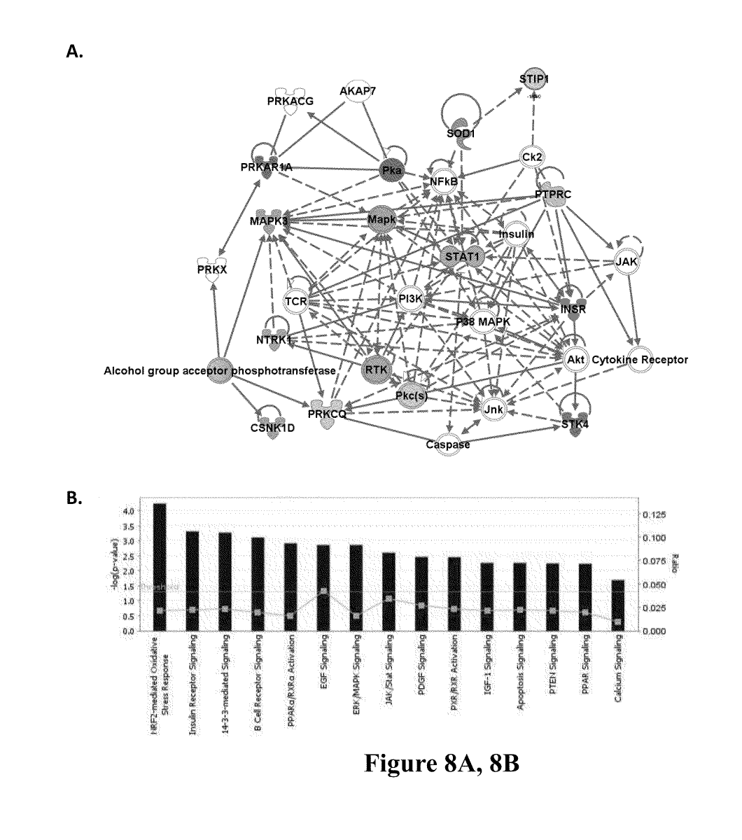

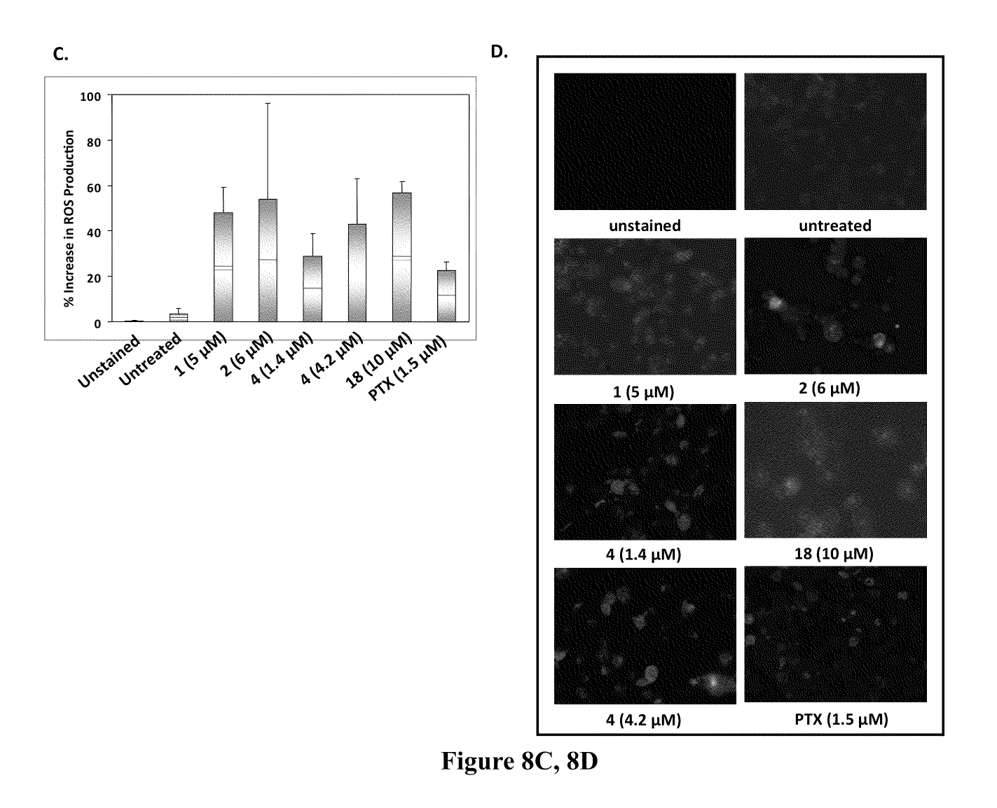

To visualize and understand the signaling networks affected by compound 1, we uploaded the sets of significantly altered pan-specific proteins to the IPA bioinformatics platform. All proteins uploaded were recognized by the IPA software as being eligible for pathway analysis. Several pathways were proposed as a result of the knowledge-based data curation (FIG. 8A). The major putative canonical pathway suggested by the IPA was the oxidative stress signaling pathway mediated by Nrf2 (nuclear factor erythroid 2-related factor 2). This pathway had a p-value of 5,82×10⁻⁵, based on the expression changes of several kinases and transcription factors identified in the Nrf2-mediated oxidative stress pathway, including protein kinase C (PKC), STIP1, MAPK3, and superoxide dismutase 1 (SOD1) (FIG. 8B).

Reactive oxygen species (ROS), such as superoxide and hydrogen peroxide, are metabolites transiently formed during normal cellular metabolism. However, elevated production of ROS damages most cellular components, and the perturbation of cellular homeostasis, in turn, results in the propagation of more ROS. The positive-feedback loop between ROS production and homeostasis disturbance ultimately causes cell death. Nrf2 is widely expressed in a number of organs and functions as a key sensor for cellular response to oxidative stress, by means of its regulatory function on the transcription of a number of genes encoding antioxidant proteins and related enzymes. SOD1 is one of two antioxidant isozymes responsible for scavenging cellular superoxide radicals and was found to be up-regulated by compound 1 in our antibody microarray assay. SOD1 is a downstream target of Nrf2, and it has been reported that Nrf2 activates the transcription of SOD1, which then phosphorylates STAT1 to execute antioxidant responses. In addition to SOD1, Nrf2 may also respond to oxidative stress by activating the mRNA transcription of STIP1. STIP1 functions as a molecular chaperone for heat shock protein 70 (HSP70) and heat shock protein 90 (HSP90). Under non-stress conditions, HSP90, acting as a molecular chaperone, facilitates protein folding and protein degradation to maintain cellular homeostasis. In addition to these functions, HSP90 was recently found to confer anti-apoptotic functions by regulating several apoptosis-related molecules. Therefore, it is conceivable that down-regulating the expression of the HSP90 protein could abolish HSP90-mediated cell survival. ROS were reported to be able to mediate HSP90 protein cleavage, and this ROS-dependent HSP90 cleavage was involved in arsenic- and MMA-induced apoptosis. On the basis of these reports, it is plausible that compound 1-induced down-regulation of STIP1 may decrease the amount of HSP90 and, hence, induce apoptosis.

While not intending to be bound by any particular theory, based on the above observations taken together with findings of the present invention, we hypothesize that compound 1 elicits cytotoxicity by inducing ROS production and further alters the expression of oxidative stress-related proteins, such as SOD1, STIP1, and HSP90. The oxidative stress sensor, Nrf2, may be one of the early proteins activated in response to compound 1 treatment.

### Compounds 1, 2, 4 and 18 Induce the Accumulation of Mitochondrial Superoxide

As a preliminary validation of the pathway proposed by IPA, we examined mitochondrial superoxide production by compounds 1, 2, 4, and 18 in MDA-MB-435 cells. Mitochondrial superoxide species was detected using MitoSOX Red mitochondrial superoxide live cell indicator. Cells were treated with these compounds at their respective IC₅₀ or 3×IC₅₀ concentrations for 24 hours. All compounds were found to induce significant amounts of superoxide upon 24 hours treatment (FIG. 8C). The flow cytometry observation was further confirmed by examining the staining pattern of mitochondrial superoxide in response to compound treatment by fluorescence microscopy. Consistently, all compounds, including the less active compound 18, remarkably enhanced the intensity of MitoSOX staining (FIG. 8D). Interestingly, paclitaxel only induced a minor amount of mitochondrial superoxide even at 1.5 μM (FIG. 8D). In addition to mitochondrial superoxide, we also measured the production of other types of reactive oxygen species using 2,7-dichlorodihydrofluorescein (DCFH) staining, and no change was observed (data not shown). Based on these observations, we propose that superoxide induction by PACMAs is a unique mechanism by which these compounds elicit cytotoxicity.

### Compound 4 and 9 Show Favorable Pharmacokinetic Profiles

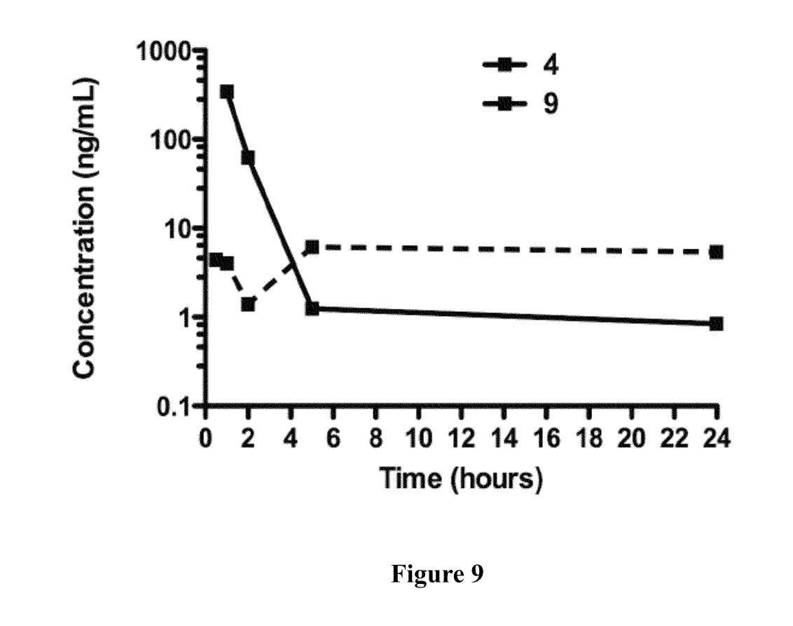

To assess the efficacy, tolerability, and pharmacokinetics of these novel compounds following i.p. administration, the pharmacokinetic profile of compounds 4 and 9 was evaluated (Table 3). Mice were treated with compounds 4 and 9 via i.p. injection and serial blood sampling was determined at indicated times after administration. The plasma concentration was determined using a validated liquid chromatography mass spectrometry (LC-MS) method where the lowest level of quantification was 1 ng/mL. One hour following i.p. administration of compounds 4 and 9, their concentrations in the blood were 344 and 6.2 ng/mL, respectively. Sequential plasma analysis revealed an apparent two compartmental pharmacokinetic elimination (FIG. 9). The half-lives of compounds 4 and 9 via i.p. administration were determined to be approximately 3.5 and 9.3 hours, respectively, when a non-compartmental pharmacokinetics analysis was used. The favorable pharmacokinetics profile of compounds 4 and 9 is consistent with the ADMET predictions described previously.

In short, the PACMAs in accordance with embodiments of the present invention are promising anticancer agents with a number of desirable features. While not intending to be bound by any particular theory, they presumably operate through an oxidative stress-mediated pathway. Of special note is their ability to show significant cytotoxicity in various drug-resistant cell lines. Their broad-spectrum in vitro cytotoxicity and in vivo efficacy point to their utility as anticancer lead compounds.

As illustrated above, the synthesis of these compounds is quite simple, amenable to parallel as well as large-scale synthesis. The following specific examples further demonstrate the various aspects and principles of the present invention.

### EXAMPLES

### Chemistry

All commercially available compounds were used without further purification. 3-Methyl-4-(1H-tetrazol-1-yl)aniline was synthesized from commercially available 2-methyl-4-nitroaniline according to the reported procedure. Flash chromatography was performed using Sorbent Technologies 60 Å silica gel (40-63 nm), All final compounds were purified to >95% purity as determined by Varian HPLC instrument with UV detection at 254 nm. Proton (¹H), fluorine (¹⁹F), and carbon (¹³C) nuclear magnetic resonance (NMR) spectra were recorded on a Varian Mercury 400 MHz NMR spectrometer. CDCl₃ (99.8% D, Cambridge Isotope Laboratories) and DMSO-d₆ (99.9% D, Cambridge Isotope Laboratories) were used in all experiments as indicated. Chemical shifts are reported as parts per million (8) relative to tetramethylsilane. CF multiplets in ¹³C NMR spectra are interpreted wherever possible.

### Synthesis of PACMA Compounds Computational

Utilizing method shown in Method 1 above, a solution of the required aniline or benzylamine A (2 mmol), aryl or heteroaryl aldehyde B (2 mmol), propynoic acid C (2.2 mmol), and isocyanide D (2.2 mmol) in methanol (2 mL) was stirred at room temperature for 48 h. The products that precipitated from the reaction mixture were filtered off, washed with aqueous methanol (1:1), and recrystallized from ethyl acetate hexane. Alternatively, the reaction mixture was evaporated to dryness and subject to column chromatography in ethyl acetatehexane. The fractions containing the target compound were evaporated to dryness, and the product E was recrystallized from ethyl acetate hexane.

### Compound 1

### N-[2-(Cyclohexylamino)-1-(4-methoxyphenyl)-2-oxoethyl]-N-(3,5-dimethoxyphenyl)Propynamide

Yield 241 mg (54%). ¹H NMR (400 MHz, CDCl₃): δ 7.07-7.13 (m, 2H), 6.71-6.77 (m, 2H), 6.30-6.45 (m, 3H), 5.86 (s, 11-1), 5.53 (br.d, J=8.3 Hz, 1H), 3.76 (s, 3H), 3.74-3.85 (m, 1H), 3.66 (s, 6H), 2.83 (s, 1H), 1.80-1.96 (m, 2H), 1.51-1.75 (m, 3H), 1.23-1.40 (m, 2H), 0.96-1.17 (m, 3H). ¹³C NMR (100 MHz, CDCl₃): δ 167.7, 160.3, 159.8, 153.5, 140.8, 131.6, 125.8, 113.9, 108.7, 101.3, 80.1, 76.1, 64.8, 55.5, 55.3, 48.8, 32.8, 25.5, 24.8, 24.7.

### Compound 2

### N-[2-(Cyclopentylamino)-1-(3-methoxyphenyl)-2-oxoethyl]-N-[3-(trifluoromethyl)phenyl]Propynamide

Yield 828 mg (93%). ¹H NMR (400 MHz, CDCl₃): δ 7.24-7.60 (m, 4H), 7.09 (t, J=7.9 Hz, 1H), 6.74 (d, J=7.9 Hz, 1H), 6.68 (d, J=7.5 Hz, 1H), 6.58 (s, 1H), 6.06 (s, 1H), 5.88 (br.d, J=7.1 Hz, 1H), 4.20 (sextet, J=6.6 Hz, 1H), 3.60 (s, 3H), 2.82 (s, 1H), 1.83-2.02 (m, ²H), 1.47-1.65 (m, 4H), 1.33-1.45 (m, 1H), 1.19-1.31 (m, 1H). ¹³C NMR (100 MHz, CDCl₃): δ 167.9, 159.5, 153.3, 139.2, 134.7, 134.5, 130.6 (quartet, J=33.1 Hz), 129.3, 128.9, 128.0 (quartet, J=3.7 Hz), 125.1 (quartet, J=3.7 Hz), 123.3 (quartet, J=273.6 Hz), 122.5, 115.2, 114.9, 81.1, 75.6, 64.1, 55.1, 51.7, 32.7, 32.6, 23.59, 23.57. ¹⁹F NMR (376 MHz, CDCl₃): δ −62.72.

### Compound 3

### N-[2-(Cyclopentylamino)-2-oxo-1-thien-2-ylethyl]-N-(3,4-dimethoxyphenyl)Propynamide

Yield 706 mg (86%). ¹H NMR (400 MHz, CDCl₃): δ 7.19 (dd, J=4.9 Hz, J=1.2 Hz, 1H), 6.91 (br.d, J=3.3 Hz, 1H), 7.19 (dd, J=4.9 Hz, J=3.7 Hz, 1H), 6.60-6.75 (m, 3H), 6.23 (br. d, J=7.5 Hz, 1H), 6.20 (s, 1H), 4.15 (sextet, J=6.6 Hz, 1H), 3.77 (s, 3H), 3.65 (s, 3H), 2.84 (s, 1H), 1.82-1.98 (m, 2H), 1.45-1.63 (m, 4H), 1.28-1.43 (m, 2H). ¹³C NMR (100 MHz, CDCl₃): δ 167.1, 153.5, 148.9, 148.2, 135.0, 131.1, 129.7, 127.8, 126.2, 122.7, 113.0, 109.9, 80.6, 75.8, 59.2, 55.7, 55.5, 51.5, 32.6, 32.5, 23.50, 23.46.

### Compound 4

### N-[2-(Cyclopentylamino)-1-(2-fluorophenyl)-2-oxoethyl]-N-[3-methyl-4-(1H-tetraazol-1-yl)phenyl]Propynamide

Yield 532 mg (60%). ¹H NMR (400 MHz, CDCl₃): δ 8.81 (s, 1H), 7.23-7.60 (m, 3H), 7.10-7.21 (m, 2H), 6.94-7.08 (m, 2H), 6.38 (s, 1H), 6.22 (br.d, J=7.1 Hz, 1H), 4.26 (sextet, J=6.6 Hz, 1H), 2.93 (s, 1H), 2.12 (s, 3H), 1.90-2.07 (m, 2H), 1.43-1.74 (m, 5H), 1.31-1.42 (m, 1H). ¹³C NMR (100 MHz, CDCl₃): 167.4, 160.7 (d, J=248.5 Hz), 153.0, 142.9, 140.5, 133.9 (d, J=14.2 Hz), 132.5, 131.2 (d, J=9.0 Hz), 131.1, 129.5, 125.6, 124.4 (d, J=2.6 Hz), 120.7 (d, J=12.9 Hz), 115.4 (d, J=21.9 Hz), 81.2, 75.6, 70.1, 57.5, 51.8, 32.72, 32.65, 23.6, 17.7. ¹⁹F NMR (376 MHz, CDCl₃): δ −115.35.

### Compound 5

### N-[2-(Cyclopentylamino)-2-oxo-1-thien-2-ylethyl]-N-(2,4-dimethoxyphenyl)Propynamide

Yield 453 mg (55%). In CDCl₃, mixture of two rotameric forms in 60:40 ratio. ¹H NMR (400 MHz, CDCl₃): δ 7.34 (d, J=8.7 Hz, 0.611), 7.12-7.22 (m, 0.6H+0.4H+0.4H), 7.01 (d, J=3.3 Hz, 0.4H), 6.91 (d, J=3.3 Hz, 0.6H), 6.86 (dd, J=5.4 Hz, J=3.7 Hz, 0.4H), 6.82 (dd, J=5.4 Hz, J=3.7 Hz, 0.6H), 6.53 (d, J=8.7 Hz, 0.4H), 6.37-6.46 (m, 0.6H+0.6H+0.4H), 6.27 (d, J=2.5 Hz, 0.6H), 6.22 (dd, J=8.7 Hz, J=2.5 Hz, 0.4H), 6.12 (s, 0.4H), 5.99 (s, 0.6H), 4.15-4.27 (m, 0.6H+0.4H), 3.82 (s, 1.2H), 3.78 (s, 1.8H), 3.75 (s, 1.2H), 3.55 (s, 1.8H), 2.74 (s, 0.4H), 2.72 (s, 0.6H), 1.89-2.06 (m, 2×0.6H+2×0.4H), 1.31-1.75 (m, 6×0.6H+6×0.4H). ¹³C NMR (100 MHz, CDCl₃): δ 167.5, 167.2, 161.2, 161.0, 157.1, 157.0, 154.7, 154.3, 135.6, 134.5, 132.0, 131.9, 129.7, 129.5, 127.7, 127.5, 126.0, 125.5, 120.9, 120.0, 104.3, 103.9, 99.0, 98.8, 78.4, 78.2, 76.2, 76.1, 60.4, 60.0, 55.7, 55.4, 55.3, 51.6, 51.3, 32.89, 32.86, 32.8, 23.8, 23.8, 23.7.

### Compound 6

### N-[2-(Cyclopentylamino)-2-oxo-1-thien-2-ylethyl]-N-(3-methoxyphenyl)Propynamide

Yield 533 mg (70%). ¹H NMR (400 MHz, CDCl₃): δ 7.23 (d, J=5.0 Hz, 1H), 7.13 (t, J=7.9 Hz, 1H), 6.96 (d, J=3.3 Hz, 1H), 6.72-6.89 (m, 4H), 6.10-6.17 (m, 2H), 4.19 (sextet, J=6.6 Hz, 1H), 3.68 (s, 31-1), 2.84 (s, 1H), 1.86-2.00 (m, 2H), 1.48-1.66 (m, 4H), 1.32-1.45 (m, 2H). ¹³C NMR (100 MHz, CDCl₃): δ 167.0, 159.5, 153.2, 139.8, 135.0, 129.9, 129.1, 128.0, 126.3, 122.2, 115.00, 114.97, 80.7, 75.7, 60.0, 55.2, 51.7, 32.7, 23.6, 23.5.

### Compound 7

### N-[2-(Cyclopentylamino)-2-oxo-1-thien-2-ylethyl]-N-(2, 5-dimethoxyphenyl)Propynamide

### The reaction was run in 2,2,2-trifluoroethanol (2 mL) for 1 h

Yield 207 mg (25%). In CDCl₃, mixture of two rotameric forms in 50:50 ratio. ¹H NMR (400 MHz, CDCl₃): δ 7.26 (br. d, J=6.2 Hz, 0.5H), 7.21 (dd, J=5.0 Hz, J=1.0 Hz, 0.5H), 7.18 (dd, J=5.0 Hz, J=1.0 Hz, 0.5H), 7.05 (d, J=2.9 Hz, 0.5H), 7.03 (d, J=3.3 Hz, 0.5H), 6.94 (d, J=3.3 Hz, 0.5H), 6.87 (dd, J=5.4 Hz, J=3.7 Hz, 0.5H), 6.73-6.86 (m, 4×0.5H), 6.69 (d, J=9.1 Hz, 0.5H), 6.51 (br.d, J=7.1 Hz, 0.5H), 6.18 (d, J=2.9 Hz, 0.5H), 6.16 (s, 0.5H), 5.89 (s, 0.5H), 4.22 (sextet, J=7.1 Hz, 0.5H), 4.20 (sextet, J=7.1 Hz, 0.5H), 3.85 (s, 3×0.5H), 3.74 (s, 3×0.5H), 3.61 (s, 3×0.5H), 3.52 (s, 3×0.5H), 2.73 (s, 0.5H), 2.70 (s, 0.5H), 1.90-2.05 (m, 4×0.5H), 1.32-1.75 (m, 12×0.5H). ¹³C NMR (100 MHz, CDCl₃): δ 167.3, 167.1, 154.2, 153.6, 153.2, 150.4, 150.2, 135.7, 134.6, 129.8, 129.5, 128.3, 127.7, 127.4, 127.2, 126.2, 125.5, 116.4, 116.2, 116.1, 115.8, 112.5, 112.0, 78.3, 78.0, 76.1, 75.9, 60.6, 60.3, 56.2, 55.8, 55.7, 55.6, 51.6, 51.2, 32.8, 32.7, 23.75, 23.71, 23.6.

### Compound 8

### N-[2-(Cyclopentylamino)-2-oxo-1-phenylethyl]-N-(2,3-dihydro-1,4-benzodioxin-6-yl)Propynamide

Yield 697 mg (86%). ¹H NMR (400 MHz, CDCl₃): δ 7.17-7.25 (m, 3H), 7.10-7.16 (m, 2H), 6.52-6.80 (m, 3H), 5.93 (s, 1H), 5.74 (d, J=7.5 Hz, 1H), 4.10-4.25 (m, 5H), 2.84 (s, 1H), 1.84-2.00 (m, 2H), 1.46-1.62 (m, 4H), 1.21-1.41 (m, 2H). ¹³C NMR (100 MHz, CDCl₃): δ 168.0, 153.9, 143.6, 142.8, 133.7, 132.1, 130.1, 128.6, 128.4, 123.8, 119.5, 116.5, 80.5, 76.1, 64.9, 64.1, 63.9, 51.5, 32.72, 32.67, 23.60, 23.57.

### Compound 9

### N-[2-(Cyclopentylamino)-2-oxo-1-phenylethyl]-N-[3-methyl-4-(1Htetraazol-1-yl)phenyl]Propynamide

Yield 461 mg (54%). 1H NMR (400 MHz, CDCl3): δ 8.81 (s, 1H), 7.13-7.35 (m, 8H), 6.13 (s, 1H), 5.96 (d, J=6.6 Hz, 1H), 4.22 (sextet, J=6.6 Hz, 1H), 2.93 (s, 1H), 2.09 (s, 1H), 1.86-2.04 (m, 2H), 1.50-1.68 (m, 4H), 1.25-1.48 (m, 2H). ¹³C NMR (100 MHz, CDCl₃): 167.9, 153.1, 142.9, 140.9, 134.4, 133.8, 133.4, 132.5, 130.1, 129.9, 129.0, 128.7, 125.5, 81.2, 75.8, 64.3, 51.7, 32.68, 32.65, 23.58, 23.56, 17.6.

### Compound 10

### N-[2-(Cyclopentylamino)-1-(2-fluorophenyl)-2-oxoethyl]-N-(2-furylmethyl)Propynamide

Yield 586 mg (80%). In CDCl₃, mixture of two rotameric forms in 70:30 ratio. ¹H NMR (400 MHz, CDCl₃): δ 7.44 (td, J=7.5 Hz, J=1.2 Hz, 0.7H), 7.29-7.37 (m, 2×0.3H), 7.22-7.29 (m, 0.7H), 7.13-7.19 (m, 2×0.3H+0.7H), 7.09 (td, J=7.5 Hz, J=1.2 Hz, 0.7H), 6.97-7.05 (m, 0.3H), 6.87-6.94 (m, 0.7H), 6.34 (s, 0.3H), 6.17 (dd, J=2.9 Hz, J=1.7 Hz, 0.3H), 6.09-6.14 (m, 0.3H+0.7H), 6.07 (s, 0.7H), 6.02 (br.d, J=7.1 Hz, 0.7H), 5.95 (d, J=3.3 Hz, 0.3H), 5.85 (d, J=3.3 Hz, 0.7H), 4.90 (d, J=17.0 Hz, 0.7H), 4.76 (d, J=17.0 Hz, 0.7H), 4.61 (d, J=15.8 Hz, 0.3H), 4.27 (d, J=15.8 Hz, 0.3H), 4.19 (sextet, J=7.1 Hz, 0.3H), 4.13 (sextet, J=7.1 Hz, 0.7H), 3.26 (s, 0.3H), 3.18 (s, 0.7H), 1.85-1.99 (m, 2×0.3H+2×0.7H), 1.47-1.64 (m, 4×0.3H+4×0.7H), 1.19-1.35 (m, 2×0.3H+2×0.7H). ¹³C NMR (100 MHz, CDCl₃): δ 167.4, 167.2, 161.4 (d, J=249.8 Hz), 161.1 (d, J=249.8 Hz), 156.3, 154.3, 154.2, 149.9, 149.8, 141.9, 141.6, 131.1, 131.0, 130.92, 130.90, 130.6 (d, J=9.0 Hz), 124.4 (d, J=2.6 Hz), 124.1 (d, J=2.6 Hz), 121.3 (d, J=14.2 Hz), 115.7 (d, J=20.6 Hz), 115.3 (d, J=21.9 Hz), 110.6, 110.3, 108.7, 108.0, 80.9, 80.0, 75.5, 74.8, 60.4, 55.23, 55.21, 51.5, 51.4, 44.6, 39.4, 32.64, 32.60, 23.6. ¹⁹F NMR (376 MHz, CDCl₃): 114.62 (0.3F), −114.75 (0.7F).

### Compound 11

### N-[2-(Cyclopentylamino)-1-(4-methoxyphenyl)-2-oxoethyl]-N-(3,5-dimethoxyphenyl)Propynamide

Yield 62 mg (7%). ¹H NMR (400 MHz, CDCl₃): δ 7.04-7.14 (m, 2H), 6.70-6.77 (m, 2H), 6.30-6.45 (m, 3H), 5.86 (s, 1H), 5.53 (br.d, J=7.1 Hz, 1H), 4.20 (sextet, J=6.6 Hz, 1H), 3.74 (s, 3H), 3.64 (s, 6H), 2.83 (s, 1H), 1.85-2.02 (m, 2H), 1.47-1.63 (m, 4H), 1.21-1.41 (m, 2H). ¹³CNMR (100 MHz, CDCl₃): δ 168.2, 160.2, 159.7, 153.4, 140.6, 131.5, 125.6, 113.8, 108.7, 101.2, 80.1, 76.1, 64.6, 55.4, 55.2, 51.6, 32.8, 32.7, 23.62, 23.60.

### Compound 12

### N-[2-(Cyclopentylamino)-1-(4-fluorophenyl)-2-oxoethyl]-N-[3-(trifluoromethyl)phenyl]Propynamide

Yield 522 mg (60%). ¹H NMR (400 MHz, CDCl₃): δ 7.24-7.54 (m, 4H), 7.09 (dd, J=8.7 Hz, J=5.4 Hz, 2H), 6.88 (t, J=8.7 Hz, 2H), 6.09 (s, 1H), 5.87 (br.s, 1H), 4.21 (sextet, J=7.1 Hz, 1H), 2.83 (s, 1H), 1.79-2.05 (m, 2H), 1.48-1.68 (m, 4H), 1.35-1.47 (m, 1H), 1.21-1.32 (m, 1H). ¹³C NMR (100 MHz, CDCl₃): δ 167.9, 162.8 (d, J=249.8 Hz), 153.4, 138.9, 134.7, 132.1 (d, J=9.0 Hz), 130.8 (quartet, J=33.5 Hz), 129.1 (d, J=2.6 Hz), 129.0, 128.1 (quartet, J=3.9 Hz), 125.3 (quartet, J=3.9 Hz), 123.3 (quartet, J=271.7 Hz), 115.6 (d, J=20.6 Hz), 81.3, 75.5, 63.3, 51.7, 31.73, 32.71, 23.63, 23.59. ¹⁹F NMR (376 MHz, CDCl₃): δ −62.80, −111.77.

### Compound 13

### N-[2-(Cyclohexylamino)-2-oxo-1-thien-2-ylethyl]-N-[3-(trifluoromethyl)phenyl]Propynamide

Yield 579 mg (67%). ¹H NMR (400 MHz, CDCl₃): δ 7.46-7.55 (m, 2H), 7.35-7.43 (m, 2H), 7.23 (dd, J=5.0 Hz, J=0.8 Hz, 1H), 6.92 (d, J=3.3 Hz, 1H), 6.85 (dd, J=5.0 Hz, J=33 Hz, 1H), 6.25 (s, 1H), 6.16 (br.d, 7.1 Hz, 1H), 4.20 (sextet, J=6.7 Hz, 1H), 2.85 (s, 1H), 1.87-2.03 (m, 2H), 1.50-1.67 (m, 4H), 1.31-1.46 (m, 2H). ¹³C NMR (100 MHz, CDCl₃): δ 167.0, 153.0, 139.1, 134.5, 134.0, 131.9 (quartet, J=33.5 Hz), 130.1, 129.1, 128.2, 127.4 (quartet, J=3.9 Hz), 126.6, 125.4 (quartet, J=3.9 Hz), 123.3 (quartet, J=273.0 Hz), 81.4, 75.4, 59.3, 51.8, 32.7, 23.63, 23.58. 19F NMR (376 MHz, CDCl3): δ 62.69.

### Compound 14

### N-[2-(Cyclopentylamino)-2-oxo-1-phenylethyl]-N-(2-naphthyl)Propynamide

Yield 652 mg (82%). ¹H NMR (400 MHz, DMSO-d₆): δ 8.24 (d, J=6.6 Hz, 1H), 7.88 (br.s, 1H), 7.74-7.84 (m, 2H), 7.60-7.73 (m, 1H), 7.40-7.50 (m, 2H), 6.99-7.17 (m, 5H), 6.10 (s, 1H), 4.11 (s, 1H), 4.08 (sextet, J=6.6 Hz, 1H), 1.71-1.90 (m, 2H), 1.38-1.68 (m, 5H), 1.21-1.33 (m, 1H). ¹³C NMR (100 MHz, DMSO-d₆): 168.2, 152.8, 136.2, 134.3, 132.2, 131.9, 130.01, 129.96, 128.7, 127.9, 127.4, 127.3, 126.5, 126.2, 83.1, 76.8, 63.6, 50.7, 32.1, 31.8, 23.44, 23.40.

### Compound 15

### N-(1,3-Benzodioxol-5-ylmethyl)-N-[2-(benzylamino)-1-(2-fluorophenyl)-2-oxoethyl]Propynamide

Yield 814 mg (92%). In DMSO-d₆, mixture of two rotameric forms in 60:40 ratio. ¹H NMR (400 MHz, DMSO-d₆): δ 8.99 (t, J=5.8 Hz, 0.4H), 8.85 (t, J=5.8 Hz, 0.6H), 7.22-7.38 (m, 6H), 7.18 (td, J=7.5 Hz, J=1.2 Hz, 0.4H), 7.11 (td, J=7.5 Hz, J=1.2 Hz, 0.6H), 6.97-7.03 (m, 0.4H), 6.84-6.91 (m, 0.6H), 6.63 (d, J=7.9 Hz, 0.6H), 6.57 (d, J=7.9 Hz, 0.4H), 6.46 (s, 0.4H), 6.42 (d, J=1.2 Hz, 0.6H), 6.34 (dd, J=7.9 Hz, −1.2 Hz, 0.6H), 6.28 (d, J=1.2 Hz, 0.4H), 6.25 (s, 0.6H), 6.19 (dd, J=7.9 Hz, J=1.2 Hz, 0.4H), 5.88-5.95 (m, 2×0.6H+2×0.4H), 5.07 (d, J=17.0 Hz, 0.6H), 4.93 (d, J=15.8 Hz, 0.4H), 4.81 (s, 0.4H), 4.62 (s, 0.6H), 4.51 (d, J=17.0 Hz, 0.6H), 4.26-4.44 (m, 2×0.6H+2×0.4H), 4.15 (d, J=15.8 Hz, 0.4H). ¹³C NMR (100 MHz, DMSO-d₆): δ 168.6, 161.4 (d, J=247.2 Hz), 161.2 (d, J=247.2 Hz), 154.7, 154.6, 151.7, 147.1, 147.0, 146.2, 146.0, 139.3, 139.2, 131.7, 131.56, 131.48, 131.40, 131.37, 131.32, 130.88, 130.86, 130.62, 130.60, 128.8, 128.7, 127.8, 127.7, 127.4, 127.3, 124.9 (d, J=2.6 Hz), 124.7 (d, J=2.6 Hz), 122.9 (d, J=14.2 Hz), 122.3 (d, J=14.2 Hz), 120.5, 119.7, 115.6 (d, J=20.6 Hz), 115.3 (d, J=20.6 Hz), 108.0, 107.9, 107.7, 106.9, 101.2, 101.1, 84.2, 83.1, 76.7, 75.9, 59.0, 54.5, 50.6, 47.4, 43.0, 42.8. ¹⁹F NMR (376 MHz, DMSO-d6): δ −114.70 (0.6F), −115.24 (OAF).

### Compound 16

### N-[2-(Benzylamino)-2-oxo-1-thien-2-ylethyl]-N-[3-(trifluoromethyl)-phenyl]Propynamide

Yield 498 mg (56%). 1H NMR (400 MHz, CDCl3): δ 7.58 (d, J=7.5 Hz, 1H), 7.53 (br.d, J=7.9 Hz, 1H), 7.47 (br. s, 1H), 7.41 (t, J=7.9 Hz, 1H), 7.22-7.35 (m, 6H), 6.95 (d, J=3.3 Hz, 1H), 6.87 (dd, J=5.0 Hz, J=3.7 Hz, 1H), 6.78 (br.t, J=5.4 Hz, 1H), 6.3 (s, 1H), 4.42-4.54 (m, 2H), 2.88 (s, 1H). ¹³C NMR (100 MHz, CDCl₃): δ 127.6, 153.0, 139.1, 137.6, 134.2, 133.9, 130.9 (quartet, J=33.5 Hz), 130.3, 129.1, 128.5, 128.3, 127.39, 127.35, 127.30, 127.26, 126.6, 125.4 (quartet, J=3.9 Hz), 123.3 (quartet, J=273.0 Hz), 81.4, 75.3, 59.6, 43.7. 19F NMR (376 MHz, CDCl₃): δ −62.57

### Compound 17

### N-(2-Chlorobenzyl)-N-[2-(cyclopentylamino)-2-oxo-1-phenylethyl]Propynamide

Yield 482 mg (61%). In CDCl₃, mixture of two rotameric forms in 80:20 ratio. ¹HNMR (400 MHz, CDCl³): δ 7.05-7.33 (m, 9×0.8H+6×0.2H), 6.97-7.03 (m, 2×0.2H), 6.87-6.92 (m, 0.2H), 6.21 (s, 0.2H), 6.06 (br.d, J=7.5 Hz, 0.2H), 5.99 (br.d, J=7.1 Hz, 0.8H), 5.78 (s, 0.8H), 5.14 (d, J=17.9 Hz, 0.8H), 4.95 (d, J=17.0 Hz, 0.2H), 4.93 (d, J=17.9 Hz, 0.8H), 4.64 (d, J=17.0 Hz, 0.2H), 4.12-4.28 (m, 0.8H+0.2H), 3.40 (s, 0.2H), 3.04 (s, 0.8H), 1.87-1.99 (m, 2×0.8H+2×0.2H), 1.49-1.65 (m, 4×0.8H+4×0.2H), 1.27-1.40 (m, 2×0.8H+2×0.2H). ¹³C NMR (100 MHz, CDCl₃): δ 167.6, 167.5, 155.0, 154.4, 134.1, 133.7, 133.5, 131.9, 131.8, 129.4, 129.17, 128.88, 128.71, 128.62, 128.56, 128.02, 127.7, 127.6, 126.3, 126.2, 81.0, 79.6, 75.5, 75.4, 66.0, 62.2, 51.5, 51.4, 48.9, 45.1, 32.6, 32.5, 23.57, 23.54.

### Compound 18

### N[2-(Cyclohexylamino)-2-oxo-1-thien-2-ylethyl]-N-phenyl Propynamide

Yield 487 mg (66%). ¹H NMR (400 MHz, CDCl₃): δ 7.16-7.35 (m, 6H), 6.97 (d, J=2.9 Hz, 1H), 6.85-6.91 (m, 1H), 6.24 (s, 1H), 6.15 (br.d, J=7.9 Hz, 1H), 3.75-3.87 (m, 1H), 2.84 (s, 1H), 1.83-1.99 (m, 2H), 1.53-1.75 (m, 3H), 1.27-1.43 (m, 2H), 1.08-1.25 (m, 3H). ¹³C NMR (100 MHz, CDCl₃): δ 166.6, 153.3, 138.7, 134.9, 130.0, 129.8, 128.8, 128.5, 127.9, 126.2, 80.8, 75.7, 59.7, 48.7, 32.5, 32.4, 25.3, 24.6, 24.5.

### Compound 19

### N-[2-(Cyclohexylamino)-1-(2-fluorophenyl)-2-oxoethyl]-N-(2-fluorophenyl)Propynamide

Yield 548 mg (69%). In CDCl₃, mixture of two rotameric forms in 75:25 ratio. 1H NMR (400 MHz, CDCl₃): δ 7.91 (td, J=7.9 Hz, J=1.7 Hz, 0.75H), 7.31 (br.t, J=7.9 Hz, 0.25H), 7.10-7.25 (m, 2×0.75H+2×0.25H), 6.84-7.09 (m, 3×0.75H+3×0.25H), 6.68-6.82 (m, 2×0.75H+2×0.25H), 6.40-6.47 (m, 0.75H+0.25H), 6.15 (s, 0.25H), 6.09 (br.d, J=7.9 Hz, 0.75H), 3.70-3.82 (m, 0.75H+0.25H), 2.80 (s, 0.25H), 2.71 (s, 0.75H), 1.49-1.98 (m, 5×0.75H+5×0.25H), 0.94-1.37 (m, 5×0.75H+5×0.25H). ¹³CNMR (100 MHz, CDCl₃): δ 167.2, 165.9, 161.0 (d, J=249.8 Hz), 160.8 (d, J=248.5 Hz), 159.2 (d, J=249.8 Hz), 159.1 (d, J=251.1 Hz), 153.8, 153.6, 133.2, 131.7, 131.5, 131.2, 130.8, 130.7, 130.65, 130.57, 126.4 (d, J=11.6 Hz), 126.0 (d, J=12.9 Hz), 124.1, 124.0 (d, J=3.9 Hz), 123.4 (d, J=3.9 Hz), 121.3 (d, J=14.2 Hz), 120.1 (d, J=14.2 Hz), 115.9 (d, J=20.6 Hz), 115.2 (d, J=20.6 Hz), 79.9, 79.2, 75.4, 75.1, 58.58, 58.56, 56.67, 56.64, 48.9, 48.4, 32.48, 32.44, 25.33, 25.27, 24.7, 24.6, 24.52, 24.48. ¹⁹F NMR (376 MHz, CDCl₃): δ −114.95 (0.75F+0.25F), −118.16 (0.25F), −119.47 (0.75F).

### Simulation to Predict the Drug-Like Properties of PACMA Compounds

After the required ligand structures of a specified configuration were built, energy minimization was performed using the Catalyst software package (Accelrys, Inc., San Diego, Calif.). The lowest-energy conformation for each compound was exported to ADMET Predictor to calculate ADME (absorption, distribution, metabolism, and excretion) properties and 3-D polar surface areas.

### Cell Cultures

The human breast cancer cell line MDA-MB-435 and the colon cancer cell line HT29 were purchased from the American Type Cell Culture (Manassas, Va.). Colon cancer cell lines HCT116 p53^{+/+} and HCT116 p53^{-/-} were kindly provided by Dr. Bert Vogelstein (Johns Hopkins Medical Institutions, Baltimore, Md.). The human ovarian carcinoma cell line (HEY), naturally resistant to cisplatin (CDDP), was kindly provided by Dr. Louis Dubeau (USC Norris Cancer Center). The bladder cancer cell lines were kindly provided by Dr. Richard Cote (USC Keck School of Medicine). Cells were maintained as monolayer cultures in the appropriate media: RPMI-1640 (HT29, HCT116 p53^{+/+}, HCT116 p53^{-/-}, and HEY cell lines) or DMEM (MDA-MB-435 and UMUC3 cell lines) supplemented with 10% fetal bovine serum (FBS) (HyClone, Road Logan, Utah) at 37° C. in a humidified atmosphere of 5% CO₂. To remove the adherent cells from the flask for subculture and counting, cells were washed with PBS without calcium or magnesium, incubated with a small volume of 1×trypsin-EDTA solution (Sigma-Aldrich, St. Louis, Mo.) for 5 min, resuspended with fresh culture medium, and centrifuged at 1200 rpm for 5 min. All experiments were performed using cells in exponential growth phase. Cells were routinely checked for Mycoplasma contamination using PlasmaTest kit (InvivoGen, San Diego, Calif.).

### Compound Dilutions

Ten millimolar stock solutions of all compounds were prepared in DMSO and stored at −20° C. Further dilutions were made fresh in cell-culture media.

### Cytotoxic Assays

Cytotoxicity was assessed by MTT assay as described previously. 43 In brief, cells were seeded in 96-well microtiter plates and allowed to attach overnight. Cells were subsequently treated with continuous exposure to corresponding drugs for 72 h. At the end of treatments, an MTT solution (at a final concentration of 0.5 mg/mL) was added to each well, and cells were incubated for 4 h at 37° C. After removal of the supernatant, DMSO was added, and the absorbance was read at 570 nm. All assays were done in triplicate. The IC₅₀ was then determined for each drug from a plot of log(drug concentration) versus percentage of cells killed.

### Colony Formation Assay

Colony formation assays were performed as previously described to confirm the activity of these compounds. In brief, cells were plated in 24-well plates at a density of 1000 cells per well and allowed to attach overnight. The next day, 1 μM concentration of each compound was added for 24 h. Cells were washed with 1×PBS, and fresh media was then added to the cells. Cells were incubated until colonies formed (810 days). Subsequently, cells were washed, fixed with a 1% glutaraldehyde solution for 30 min, and stained with a solution of crystal violet (2%) for 30 min. After staining, cells were thoroughly washed with distilled water. Colonies were imaged on a Chemi-Doc Imaging System (Bio-Rad, Hercules, Calif.) and counted using the Quantity One software package (Bio-Rad, Hercules, Calif.). The data reported is a representative of at least three independent experiments.

### In Vivo Mouse Xenograft Studies

Sixteen virgin female athymic nude (nu/nu) mice (Simonsen Laboratory Inc., Gilroy, Calif.) were used for in vivo efficacy testing. The animals were fed ad libitum and kept in temperature controlled rooms at 20±2° C. with a 12 h lightdark period. Animal care and manipulation were in agreement with the University of Southern California (USC) institutional guidelines, which are in accordance with the Guidelines for the Care and Use of Laboratory Animals.

Human breast cancer MDA-MB-435 cells in logarithmic phase growth from in vitro cell culture were inoculated subcutaneously on the rear flank of the mice (1.5×10⁶ cells/mouse) under aseptic conditions. Tumor growth was assessed twice weekly by measuring tumor diameters with a Vernier caliper (length×width). Tumor weight was calculated according to the formula: TW (mg)=tumor volume (mm³)=d²×D/2, where d and D are the shortest and longest diameters, respectively. Tumors were allowed to grow to an average volume of 100 mm³. Animals were then randomly assigned to control and treatment groups (n=8), to receive vehicle control or compound 2 (10 mg/kg for 9 days, 20 mg/kg for 5 days, and 40 mg/kg for 12 days, 2 was dissolved in 10% DMSO/90% sesame oil) via i.p. injection once daily. Treatment of each animal was based on the average body weight of the treatment group.

### Statistical Analysis

Eight mice were assigned to each group and the results were expressed as the mean±SEM. Statistical analysis and p-value determination were done by two-tailed paired t-test with a confidence interval of 95% for determination of the differences between groups. A p-value of <0.05 was considered to be statistically significant. ANOVA was used to test for significance among groups. The SAS statistical software package (SAS Institute, Cary, N. C.) was used for statistical analysis.

### Cell Cycle Analysis

Cell cycle perturbations were analyzed by propidium iodide DNA staining. Exponentially growing MDA-MB-435 cells were treated with 5 μM compound 4 for 6, 12, and 24 h. At the end of treatment, cells were collected and washed with 1×PBS after a gentle centrifugation at 1500 rpm for 5 min. Cells were then thoroughly resuspended in 0.5 mL of PBS and fixed in 70% ethanol overnight at 4° C. Ethanol-fixed cells were centrifuged at 3000 rpm for 5 min and washed twice in PBS to remove residual ethanol. For cell cycle analysis, the pellets were resuspended in 1 mL of PBS containing 0.02 mg/mL of propidium iodide and 0.5 mg/mL of DNase-free RNase A and incubated at 37° C. for 30 min. Cell cycle profiles were obtained using a BD LSRII flow cytometer (BD Biosciences, San Jose, Calif.). Propidium iodide was excited using a 488 nm blue argon laser. Resulting fluorescence was measured using a PMT detector equipped with 550 nm long pass dichroic mirror and 575/26 bandpass filter. Data were analyzed with the ModFit LT software package (Verify Software House, Inc., Topsham, Me.).

### Apoptosis Assay

To quantify drug-induced apoptosis, annexin V/propidium iodide staining was performed and analyzed by flow cytometry. MDA-MB-435 cells were treated with 10 μM compound 1, 2, or 4 for 24 and 48 h. After treatment, both floating and adherent cells were combined and subjected to annexin V/propidium iodide staining using an annexin V-FITC apoptosis detection kit (Oncogene Research Products, San Diego, Calif.) according to manufacturer's recommendations. Untreated control cells (48 h) were maintained in parallel to drugtreated groups. Double staining was used to distinguish between viable, early apoptotic, and necrotic or late apoptotic cells. The resulting fluorescence (FITC-A channel for green fluorescence and PE-A channel for red fluorescence) was measured by flow cytometry using a BD LSRII flow cytometer (BD Biosciences, San Jose, Calif.). According to this method, the lower left quadrant shows the viable cells, the upper left quadrant shows early apoptotic cells, the upper right quadrant shows late apoptotic, and the lower right quadrant shows both necrotic cells and cellular debris.

### Western Blotting Analysis

Cells were detached using 1×trypsin-EDTA solution and collected by centrifugation at 1200 rpm for 5 min. Cells were then lysed in 80 μL of 1× cell lysis buffer (50 mM Tris-HCl, pH 7.5, 150 mM NaCl, and 1% NP-40) and pelleted by centrifugation at 14,000 rpm for 20 min at 4° C. Protein concentration of the whole cell lysates was measured using BCA protein assay and equal amounts of total protein were resolved on a 10% tris-glycine gel via SDS-PAGE. The separated proteins were electro-blotted onto nitrocellulose membrane and blocked in 5% BSA/PBS for 1 h at room temperature. The membrane was probed with anti-p53 (SC-126, Santa Cruz Biotechnology Inc.), anti-caspase 9 (Cat. no. 9508; Cell Signaling Technology), anti-a-tubulin (SC-5286, Santa Cruz Biotechnology Inc.), and anti-GAPDH(Y3322GAPDH, Biochain Institute, Inc.) antibodies at 4 C overnight. Horseradish peroxidase-conjugated secondary antibodies (Invitrogen, Carlsbad, Calif.) in combination with SuperSignal Dura (ThermoFisher, Rockford, Ill.) enhanced chemiluminescence (ECL) solutions were used to visualize proteins of interest with a ChemiDoc Imaging system (Bio-Rad, Hercules, Calif.).

### Kinexus Antibody Microarray

MDA-MB-435 cells were treated with 0.1 μM compound 1 for 24 h. After treatment, cells were washed with ice-cold PBS to remove residual medium. Cells were then lysed in 200 μL of lysis buffer (20 mM MOPS, pH 7.0, 2 mM EGTA, 5 mM EDTA, 30 mM sodium fluoride, 60 mM β-glycerophosphate, pH 7.2, 20 mM sodium pyrophosphate, 1 mM sodium orthovanadate, 1 mM phenylmethylsulfonylfluoride, 3 mM benzamidine, 5 μM pepstatin A, 10 μM leupeptin, 1% Triton X-100, 1 mM dithiothreitol) and collected into a microcentrifuge tube. The cell lysates were sonicated four times for 10 s each with 15 s intervals on ice to rupture cell membrane and shear chromosomal DNA. After sonication, the homogenates were centrifuged at 90,000×g for 30 min at 4° C. The supernatants were then transferred to a cleanmicrocentrifuge tube, and the protein concentrations were measured using BCA protein assay. Aliquots (250 μL) of the whole cell lysates were submitted to Kinexus for the 628-antibody microarray analysis.

### Ingenuity Pathway Analysis

Potential signaling pathways induced by compound 1 were analyzed by Ingenuity Pathway Analysis (IPA) software using the Kinexus 628-antibody microarray results. The statistically significant up-regulated or down-regulated pan-specific proteins with their corresponding Swiss-Prot accession numbers and ratio changes were uploaded as an Excel spreadsheet file to the IPA server. Compound 1-mediated signaling pathways were then analyzed by IPA core analysis.

### Detection of Mitochondrial Superoxide Production

MDAMB-435 cells were treated with compounds 1, 2, 4, and 18 at their IC₅₀ or 3×IC₅₀ for 24 h. After drug treatments, a solution of 5 μM MitoSOX Red mitochondrial superoxide indicator (Invitrogen, Carlsbad, Calif.) was added, and cells were incubated for 10 min at 37° C. Cells were then washed three times with Hank's balanced salt solution (HBSS) to remove excess MitoSOX. After washing, cells were either observed under the fluorescence microscope or collected by trypsinization, washed twice with HBSS, and measured for superoxide production using a BD LSRII flow cytometer (BD Biosciences, San Jose, Calif., USA). Data were analyzed with the BD FACSDiva software package (BD Biosciences, San Jose, Calif., USA).

### Pharmacokinetics Study

Preliminary pharmacokinetics of compounds 4 and 9 were performed using 18 mice, each time point consisting of two mice. Mice were stratified to various doses given as an i.p. injection of compounds 4 and 9 (24 mg/kg). At the specified time points following drug administration, blood was collected with heparinized syringes. The samples were allowed to settle in 500 μL eppendorf vials and then centrifuged at 1500 rpm for 10 min. The plasma was removed, transferred into new eppendorf tube, and frozen at −80° C. until analysis.

Although the present invention has been described in terms of specific exemplary embodiments and examples, it will be appreciated that the embodiments disclosed herein are for illustrative purposes only and various modifications and alterations might be made by those skilled in the art without departing from the spirit and scope of the invention as set forth in the following claims.

TABLE 1
Physicochemical Properties of PACMA Compounds
Calculated by Computational Simulation*

| compound | MW | MLogP | S+ logP | PSA | HBD | HBA |
|---|---|---|---|---|---|---|
| 1 | 450.5 | 2.54 | 3.62 | 55.18 | 1 | 7 |
| 2 | 444.5 | 3.70 | 4.20 | 40.65 | 1 | 5 |
| 3 | 412.5 | 2.26 | 2.83 | 49.91 | 1 | 6 |
| 4 | 446.5 | 3.91 | 2.81 | 122.06 | 1 | 7 |
| 5 | 412.5 | 2.26 | 2.86 | 44.69 | 1 | 6 |
| 6 | 382.5 | 2.55 | 2.99 | 49.85 | 1 | 5 |
| 7 | 412.5 | 2.26 | 2.74 | 45.72 | 1 | 6 |
| 8 | 404.5 | 2.62 | 2.97 | 51.90 | 1 | 6 |
| 9 | 428.5 | 3.54 | 2.63 | 129.21 | 1 | 7 |
| 10 | 368.4 | 2.39 | 2.27 | 43.08 | 1 | 5 |
| 11 | 436.5 | 2.33 | 3.23 | 54.79 | 1 | 7 |
| 12 | 432.4 | 4.38 | 4.67 | 32.99 | 1 | 4 |
| 13 | 434.5 | 3.88 | 4.53 | 39.73 | 1 | 4 |
| 14 | 410.5 | 4.11 | 4.93 | 37.34 | 1 | 4 |
| 15 | 444.5 | 3.32 | 3.41 | 57.05 | 1 | 6 |
| 16 | 442.5 | 3.86 | 4.26 | 42.75 | 1 | 4 |
| 17 | 429.3 | 4.11 | 4.11 | 27.71 | 1 | 4 |
| 18 | 366.5 | 3.07 | 3.35 | 31.53 | 1 | 4 |
| 19 | 396.5 | 4.17 | 4.24 | 31.57 | 1 | 4 |
| camptothecin | 348.4 | 1.67 | 1.60 | 103.63 | 1 | 5 |
| doxorubicin | 543.5 | −0.82 | 0.54 | 233.93 | 6 | 12 |
| paclitaxel | 839.9 | 0.54 | 3.66 | 156.48 | 4 | 15 |

*MW, molecular weight; MLogP, Moriguchi octane-water partition coefficient; S+ logP, SimulationPlus, Inc., model of logP; PSA, three-dimensional polar surface area; HBD, hydrogen-bond donor; HBA, hydrogen-bond acceptor.

TABLE 2
In vitro cytotoxicity of compounds 1-19 in a panel of human cancer cell lines

| | | | | | | | | |
|---|---|---|---|---|---|---|---|---|
| # | HT29 | HCT116 p53^{+/+} | HCT116 p53^{-/-} | HEY | MDA-MB-435 | UMUC3 | 5637 | NCI/ADR-RES |

IC₅₀* (μM)

| | | | | | | | | |
|---|---|---|---|---|---|---|---|---|
| 1 | 6.3 ± 1.5 | 12.5 ± 2.1 | 11.0 | 4.4 ± 3.0 | 5.4 ± 2.9 | >20 | 2.2 ± 0.2 | 0.3 ± 0.0 |
| 2 | 7.2 ± 0.2 | >10 | 8.8 ± 0.4 | 5.2 ± 3.4 | 6.3 ± 1.1 | >20 | 0.6 ± 0.4 | 0.2 ± 0.1 |
| 3 | 5.5 ± 4.2 | 7.7 ± 1.9 | 9.6 ± 4.9 | 3.4 ± 1.5 | 2.0 ± 0.1 | >20 | 2.0 ± 0.7 | 0.9 ± 0.8 |
| 4 | 1.8 ± 1.7 | 6.3 ± 1.8 | 5.5 ± 0.7 | 1.5 ± 0.8 | 1.4 ± 0.2 | 6.3 ± 1.0 | 0.7 ± 0.5 | 1.0 ± 0.3 |
| 5 | 2.3 ± 0.3 | 6.9 ± 0.6 | 7.0 ± 0.7 | 4.1 ± 3.4 | 1.7 ± 0.5 | >20 | 1.7 ± 0.8 | 0.4 ± 0.0 |
| 6 | 2.3 ± 0.3 | 7.2 ± 0.2 | 5.6 ± 0.8 | 1.9 ± 0.7 | 1.7 ± 0.4 | 6.9 ± 1.2 | 0.3 ± 0.0 | 0.3 ± 0.0 |
| 7 | 1.6 ± 1.0 | 5.7 ± 0.6 | 4.7 ± 2.3 | 1.5 ± 1.1 | 0.5 ± 0.3 | 5.7 ± 1.5 | 0.3 ± 0.0 | 0.3 ± 0.0 |
| 8 | 3.2 ± 1.2 | 17.3 ± 1.1 | >20 | 4.1 ± 3.4 | 1.8 ± 0.6 | >20 | 1.9 ± 0.6 | 0.3 ± 0.0 |
| 9 | 1.5 ± 1.1 | 7.2 ± 0.2 | 5.7 ± 0.9 | 1.9 ± 0.3 | 0.6 ± 0.3 | 7.8 ± 1.8 | 0.4 ± 0.1 | 1.0 ± 0.7 |
| 10 | >10 | 10.5 | 13.0 ± 2.8 | 4.3 ± 3.9 | 1.7 ± 0.6 | >20 | 2.9 ± 0.8 | 1.2 ± 0.4 |
| 11 | 2.1 ± 0.4 | 15.0 | >20 | 2.3 ± 0.4 | 2.4 ± 0.1 | >20 | 1.9 ± 0.6 | 0.3 ± 0.0 |
| 12 | 4.8 ± 3.2 | 7.5 ± 0.7 | 6.9 ± 0.6 | 2.4 ± 0.2 | 5.2 ± 1.2 | 8.0 ± 1.4 | 0.5 ± 0.3 | 0.4 ± 0.0 |
| 13 | 6.0 ± 1.4 | 8.3 ± 1.1 | 10.4 ± 0.2 | 5.2 ± 3.6 | 6.9 ± 0.8 | >20 | 1.6 ± 0.7 | 0.4 ± 0.0 |
| 14 | 4.9 ± 3.4 | 6.8 ± 1.8 | 7.4 ± 0.8 | 1.7 ± 1.1 | 7.3 ± 0.0 | >20 | 0.5 ± 0.3 | 0.3 ± 0.0 |
| 15 | >10 | >10 | >20 | >10 | >10 | >20 | 2.8 ± 1.1 | 1.3 ± 1.0 |
| 16 | 7.3 ± 0.3 | 9.0 ± 1.4 | 16.1 ± 5.6 | 7.1 ± 2.6 | 4.2 ± 3.0 | >20 | 1.2 ± 1.1 | 0.7 ± 0.6 |
| 17 | 5.4 ± 3.0 | 7.7 ± 0.5 | 7.3 ± 1.4 | 5.8 ± 1.3 | 8.2 ± 0.2 | >20 | 1.8 ± 0.1 | 2.2 ± 0.2 |
| 18 | >10 | 14.0 | >20 | >10 | >10 | >20 | 3.5 ± 1.4 | 1.3 ± 0.4 |
| 19 | 7.7 ± 0.5 | 11.9 ± 0.1 | 10.1 ± 2.9 | 5.6 ± 2.1 | 6.5 ± 2.1 | >20 | 2.1 ± 0.0 | 0.4 ± 0.1 |

*Cytotoxic concentration (IC₅₀) is defined as drug concentration causing a 50% decrease in cell population using MTT assay as described in the experimental section.

TABLE 3
PK parameters of compounds 4 and 9 after I.P. administration

| PK Parameters | Compound 4 | Compound 9 |
|---|---|---|
| Cmax (ng/mL) | 344 | 6.2 |
| Tₘₐₓ (h) | 1.0 | 5 |
| Kₑₗ (h⁻¹) | 0.206 | 0.0 |
| t_{1/2} (h) | 3.5 | 9.3 |
| AUC (ng*h/mL) | 520 | 124.9 |

TABLE 4
In vitro cytotoxicity of selected compounds in a
panel of human ovarian cancer cell lines

| | IC₅₀ (μM)^{a} | | | |
|---|---|---|---|---|
| | | NCI/ADR- | | |
| Compound | OVCAR8 | RES | HEY | OVCAR3 |
| 20 | 0.2 | 1.3 | NT^{b} | NT |
| 21 | 2.2 | 0.52 | 0.31 | 3.1 |
| 22 | 1.5 | 0.32 | 0.33 | 2.5 |
| 23 | 1.5 | 2 | 0.72 | 0.25 |
| 24 | 5 | 0.45 | NT | 0.6 |
| 25 | 2.2 | 0.32 | NT | 0.53 |
| 26 | 2.2 | 0.32 | NT | 1.5 |
| 27 | 2.2 | 0.33 | NT | 0.42 |
| 28 | 0.52 | 0.02 | NT | 0.23 |
| 29 | 0.5 ± 0.3 | NT | NT | NT |
| 30 | 10 | 1.8 | NT | 3 |
| 31 | 1.5 | 2.5 | NT | 2.5 |
| 32 | 4.3 | 2.2 | NT | 2.5 |
| 33 | 1 | 0.43 | NT | 0.43 |
| 34 | 1.2 | 2.5 | NT | 2.5 |
| 35 | 2.9 | 5.5 | NT | 7.5 |
| 36 | 3.1 | 8.5 | NT | 2.3 |
| 37 | 1.5 | 9 | NT | 2.3 |
| 38 | 2.8 ± 0.7 | 2.6 ± 1.5 | NT | 0.32 |
| 39 | 6 | 8 | NT | >10 |
| 40 | 10.4 ± 1.6 | 12.8 ± 3.8 | NT | NT |
| 41 | 0.4 ± 0.2 | NT | NT | NT |
| 42 | 3.4 ± 0.4 | NT | NT | NT |
| 43 | 2.2 | 0.14 | NT | 0.3 |
| 44 | >10 | 6.3 | NT | >10 |
| 45 | 5 | >10 | 0.32 | NT |
| 46 | 0.5 | NT | NT | NT |

^{a}IC₅₀ is defined as drug concentration causing a 50% decrease in cell population.
^{b}NT, not-tested.

### REFERENCES

The entire disclosure of each reference cited herein or listed below is relied upon and incorporated by reference herein.
1. Dy, G. K.; Adjei, A. A Systemic cancer therapy: evolution over the last 60 years. Cancer 2008, 113, 1857-1887.
2. Kummar, S.; Chen, H. X.; Wright, J.; Holbeck, S.; Millin, M. D.; Tomaszewski, J.; Zweibel, J.; Collins, J.; Doroshow, J, H. Utilizing targeted cancer therapeutic agents in combination: novel approaches and urgent requirements. Nat Rev Drug Discov 2010, 9, 843-856.
3. Neamati, N.; Barchi, J. J., Jr. New paradigms in drug design and discovery. Curr Top Med Chem 2002, 2, 211-227.
4. Loging, W.; Harland, L.; Williams-Jones, B, High-throughput electronic biology: mining information for drug discovery. Nat Rev Drug Discov 2007, 6, 220-230.
5. Lamb, J. The Connectivity Map: a new tool for biomedical research. Nat Rev Cancer 2007, 7, 54-60.
6. Akritopoulou-Zanze, 1. Isocyanide-based multicomponent reactions in drug discovery. Curr Opin Chem BioI 2008, 12, 324-331.
7. Akritopoulou-Zanze, I.; Djuric, S. W. Recent advances in the development and applications ofpost-ugi transformations. Heterocycles 2007, 73, 125-147.
8. Domling, A Recent developments in isocyanide based multicomponent reactions in applied chemistry. Chem Rev 2006, 106, 17-89.
9. Lipinski, C. A. Drug-like properties and the causes of poor solubility and poor permeability. J Pharmacal Toxicol Methods 2000, 44, 235-249.
10. Lipinski, C. A; Lombardo, F.; Dominy, B. W.; Feeney, P. J. Experimental and computational approaches to estimate solubility and permeability in drug discovery and development settings. Adv Drug Deliv Rev 2001, 46, 3-26.
11. Palm, K.; Luthman, K.; Ungell, A L.; Strandlund, G.; Artursson, P. Correlation of drug absorption with molecular surface properties. J Pharm Sci 1996, 85, 32-39.
12. Palm, K.; Stenberg, P.; Luthman, K.; Artursson, P. Polar molecular surface properties predict the intestinal absorption of drugs in humans. Pharm Res 1997, 14, 568-571.
13. Palm, K.; Lutbman, K.; Ungell, A. L.; Strandlund, G.; Beigi, F.; Lundahl, P.; Artursson, P. Evaluation of dynamic polar molecular surface area as predictor of drug absorption: comparison with other computational and experimental predictors. J Med Chem 1998, 41, 5382-5392.
14. Kelder, J.; Grootenhuis, P. D.; Bayada, D. M.; Delbressine, L. P,; Ploemen, J. P. Polar Molecular Surface as a Dominating Deterrnivant for Oral Absorption and Brain Penetration of Drugs Pharmaceutical Research 1999, 16, 6.
15. Alvarez, M.; Paull, K.; Monks, A.; Hose, C.; Lee, J. S.; Weinstein, J.; Greyer, M.; Bates, S.; Fojo, T. Generation of a drug resistance profile by quantitation of mdr-IIP-glycoprotein in the cell lines of the National Cancer Institute Anticancer Drug Screen., J Clin Invest 1995, 95, 2205-2214.
16. Lee, J. S.; Paull, K.; Alvarez, M.; Hose, C.; Monks, A.; Greyer, M.; Fojo, A. T.; Bates, S. E. Rhodamine efflux patterns predict P-glycoprotein substrates in the National Cancer Institute drug screen. Mol Pharmacol 1994, 46, 627-638.
17. Sanchez-Carbayo, M.; Socci, N. D.; Charytonowicz, E.; Lu, M.; Prystowsky, M.; Childs, G.; Cordon-Cardo, C. Molecular profiling of bladder cancer using cDNA microarrays: defining histogenesis and biological phenotypes. Cancer Res 2002, 62, 6973-6980.
18. Dehnavi, E.; Soheili, Z. S.; Sarniei, S.; Ataei, Z.; Aryan, H. The effect of TGF-beta2 on MMP-2 production and activity in highly metastatic human bladder carcinoma cell line 5637. Cancer Invest 2009, 27, 568-5674.
19. Arima, Y.; Inoue, Y.; Shibata, T.; Hayashi, H.; Nagano, 0.; Saya, H.; Taya, Y. Rb depletion results in deregulation of E-cadherinand induction of cellular phenotypic changes that are characteristic of the epithelial-to-mesenchymal transition. Cancer Res 2008, 68, 5104-5112.
20. Schnier, J. B.; Nishi, K.; Goodrich, D. W.; Bradbury, E. M. G1 arrest and down-regulation of cyclin E/cyclin-dependent kinase 2 by the protein kinase inhibitor staurosporine are dependent on the retinoblastoma protein in the bladder carcinoma cell line 5637. Proc Natl Acad Sci USA 1996, 93, 5941-5946.
21. Lowe, S. W.; Lin, A. W. Apoptosis in cancer. Carcinogenesis 2000, 21, 485-495.
22, Assuncao Guimaraes, C.; Linden, R. Programmed cell deaths. Apoptosis and alternative deathstyles. Eur J Biochern 2004, 271, 1638-1650.
23. Pommier, Y.; Sordet, 0.; Antony, S.; Hayward, R. L.; Kohn, K. W. Apoptosis defects and chemotherapy resistallce: molecular interaction maps and networks. Oncogene 2004, 23, 2934-2949.
24. Norbury, C. J.; Zhivotovsky, B. DNA damage-induced apoptosis. Oncogene 2004, 23, 2797-2808.
25. Zhang, J. Y. Apoptosis-based anticancer drugs. Nat Rev Drug Discov 2002, 1, 101-102.
26. Schuler, M.; Bossy-Wetzel, E.; Goldstein, J. C.; Fitzgerald, P.; Green, D. R. p53 induces apoptosis by caspase activation tbrough mitochondrial cytochrome c release. J Biol Chem 2000, 275, 7337-7342.
27. Ding, H. F.; McGill, G.; Rowan, S.; Schmaltz, C.; Shimamura, A.; Fisher, D. E. Oncogenedependent regulation of caspase activation by p53 protein in a cell-free system. J BioI Chern 1998, 273, 28378-28383.
28. Kobayashi, M.; Yamamoto, M. Molecular mechanisms activating the Nrf2-Keapl pathway of antioxidant gene regulation. Antjoxid Redox Signal 200S, 7, 385-394.
29. Zhang, D. D. The Nrf2-Keap 1-ARE signaling pathway: The regulation and dual function of Nrf2 in cancer. Antioxid Redox Signal 2010, 13, 1623-1626.
30. Giudice, A.; Arra, C.; Turco, M. C. Review of molecular mechanisms involved in the activation of the Nrt2-ARE signaling pathway by chemopreventive agents. Methods Mol BioI 2010, 647, 37-74.
31. Aieksunes, L. M.; Manautou, J. E. Emerging role of Nrf2 in protecting against hepatic and gastrointestinal disease. Toxicol Pathol 2007, 35, 459-473.
32, Wasserman, W. W.; Fahl, W. E. Functional antioxidant responsive elements. Proc Natl Acad Sci USA 1997, 94, 5361-5366.
33. Rushmore, T. H.; Mortou, M. R.; Pickett, C. B. The antioxidant responsive element. Activation by oxidative stress and identification of the DNA consensus sequence require for functional activity. J Biol Chem 1991, 266, 11632-11639.
34. Friling, R. S.; Bensimon, A.; Tichauer, Y.; Daniel, V. Xenobiotic-inducible expression of murine glutathione S-transferase Ya subunit gene is controlled by an electrophileresponsive element. Proc Natl Acad Sci USA 1990, 87, 6258-6262.
35. Chen, C. W.; Chang, Y. H.; Tsi, C. J.; Liu, W. W. Inhibition of IFN-gamma-mediated inducible nitric oxide synthase induction by the peroxisome proliferator-activated receptor gamma agonist, 15-deoxy-delta 12,14-prostaglandin J2, involves inhibition of the upstream Janus kinase/STAT! signaling pathway. J Immunol 2003, 171, 979-988.
36. Kwak, M. K.; Wakabayashi, N.; Itoh, K.; Motohashi, H.; Yamamoto, M.; Kensler, T. W. Modulation of gene expression by cancer chemopreventive dithiolethiones through the Keapl-Nrf2 pathway. Identification of novel gene clusters for cell survival. J Biol Chem 2003, 278, 8135-8145.
37. Zhang, 1.; Nephew, K. P.; Gallagher, P. J. Regulation of death-associated protein kinase. Stabilization by HSP90 heterocomplexes. J Biol Chem 2007, 282, 11795-11804.
38. Pespeni, M. H.; Hodnett, M.; Abayasiriwardana, K. S.; Roux, 1; Howard, M.;. Broaddus, V. C.; Pittet, J. F. Sensitization of mesothelioma cells to tumor necrosis factor-related apoptosis-inducing ligand-induced apoptosis by heat stress via the inhibition of the 3-phosphoinositide-dependent kinase 1IAkt pathway. Cancer Res 2007, 67, 2865-2871.
39. Shen, S. C.; Yang, 1. Y.; Lin, H. Y.; Wu, C. Y.; Suo T. H.; Chen, Y. C. Reactive oxygen species-dependent HSP90 protein cleavage participates in arsenical As(+3)- and MMA(+3)-induced apoptosis through inhibition oftelomerase activity via JNK activation. Toxicol Appl Pharmacol 2008, 229, 239-251.
40. Gaponik, P. N.; Karavai, V. P.; Dayshko, I. E.; Degtyarik, M. M.; Bogatikov, A. N, Synthesis and properties of phenylenebis-1H-tetrazoles Chem Heterocycl Compd 1990, 26, 1274-1278.
41. Buick, R. N.; Pullano, R; Trent, J. M. Comparative properties of five human ovarian adenocarcinoma cellines. Cancer Res 1985, 45, 3668-3676.
42. Hamaguchi, K.; Godwin, A. K.; Yakushiji, M.; O'Dwyer, P. 1; Ozols, R F.; Hamilton, T. C. Cross-resistance to diverse drugs is associated with primary cisplatin resistance in ovarian cancer cell lines. Cancer Res 1993, 53, 5225-5232.
43. Carmichael, 1; DeGraff, W. G.; Gazdar, A. F.; Minna, J. D.; Mitchell, J. B. Evaluation of a tetrazolium-based semiautomated colorimetric assay: assessment of chemosensitivity testing. Cancer Res 1987, 47, 936-942.
44. Munshi, A.; Hobbs, M.; Meyn, R E. Clonogenic cell survival assay. Methods Mol Med 2005, 110, 21-28
45. Yamada, R.; Cao, X.; Butkevich, A. N.; Millard, M.; Odde, S.; Mordwinkin, N.; Gundla, R.; Zandi, E.; Louie, S. G.; Petasis, N. A.; Neamati, N. Discovery and preclinical evaluation of a novel cass of cytotoxic propynoic acid carbamoyl methyl amides (PACMAs). J. Med. Chem. 2011, 54, 2902-2914.

What is claimed is:

1. A compound having the general formula: 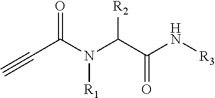
wherein:
R₁ is hydrogen, alkyl, cycloalkyl, aryl, or heteroaryl;
R₂ is hydrogen, alkyl, cycloalkyl, aryl, or heteroaryl; and
R₃ is hydrogen, alkyl, aryl, benzyl, or cycloalkyl.

2. A compound having the general formula: 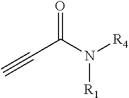
wherein:
R₁ is hydrogen; and
R₄ is an alkyl, cycloalkyl, aryl, or heteroaryl.

3. A compound having the following general formula: 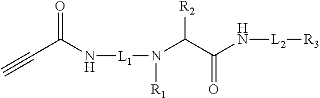
wherein:
R₁ is hydrogen, alkyl, cycloalkyl, aryl, or heteroaryl;
R₂ is hydrogen, alkyl, cycloalkyl, aryl, or heteroaryl;
R₃ is hydrogen, alkyl, aryl, or cycloalkyl; and
L₁ and L₂ are linker groups independently selected from the group consisting of alkyl, cycloalkyl, aryl, and heteroaryl.

4. A compound according to claim 1, wherein said compound further contains a substituent capable of being used in a biological or medical diagnostic imagining technique to quantify or identify associated biomarker proteins.

5. A compound according to claim 3, wherein said compound further contains a substituent capable of being used in a biological or medical diagnostic imagining technique to quantify or identify associated biomarker proteins.

6. A compound having any of the formulas:

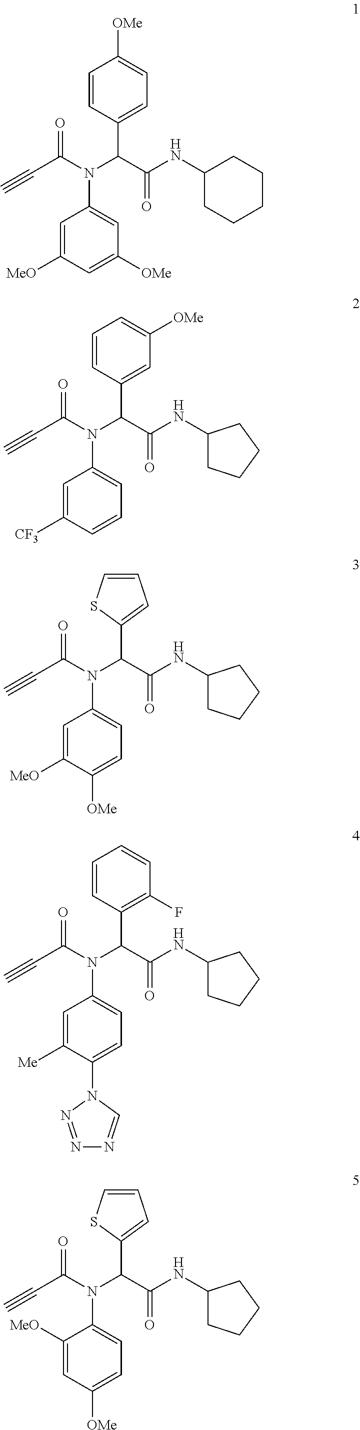

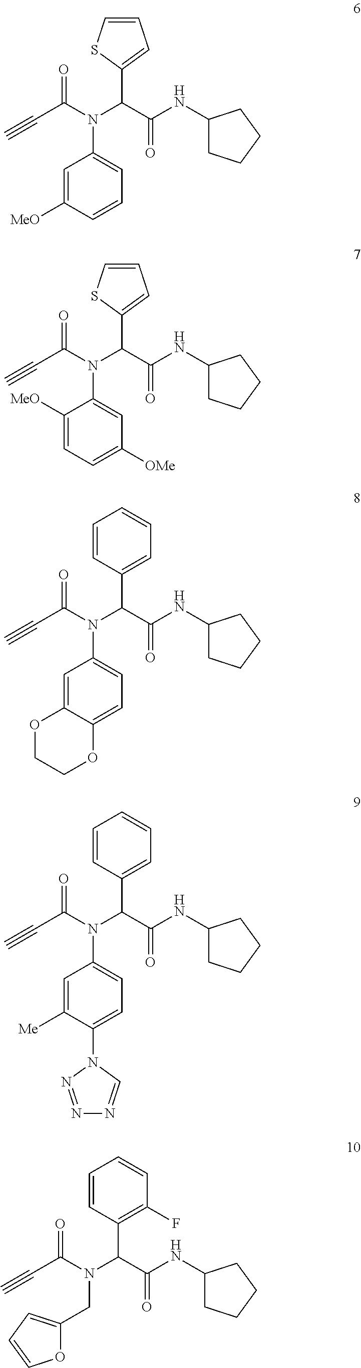

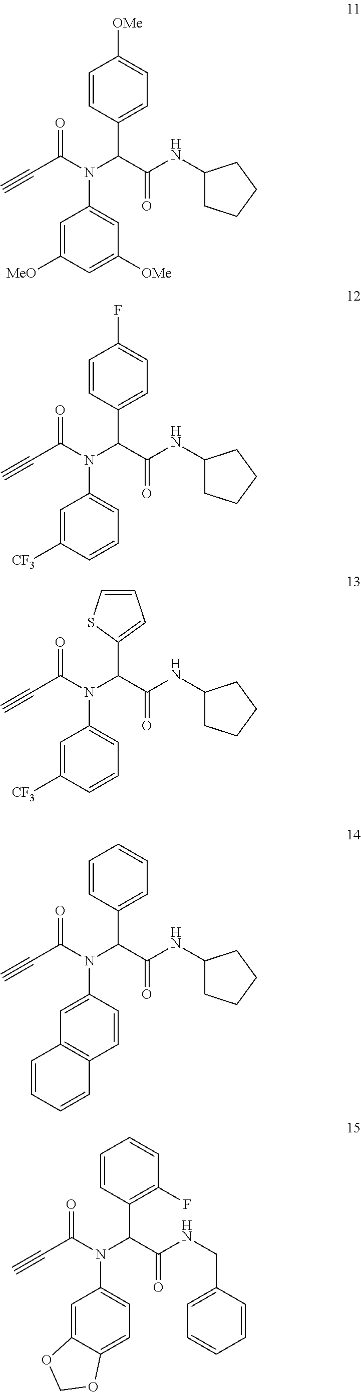

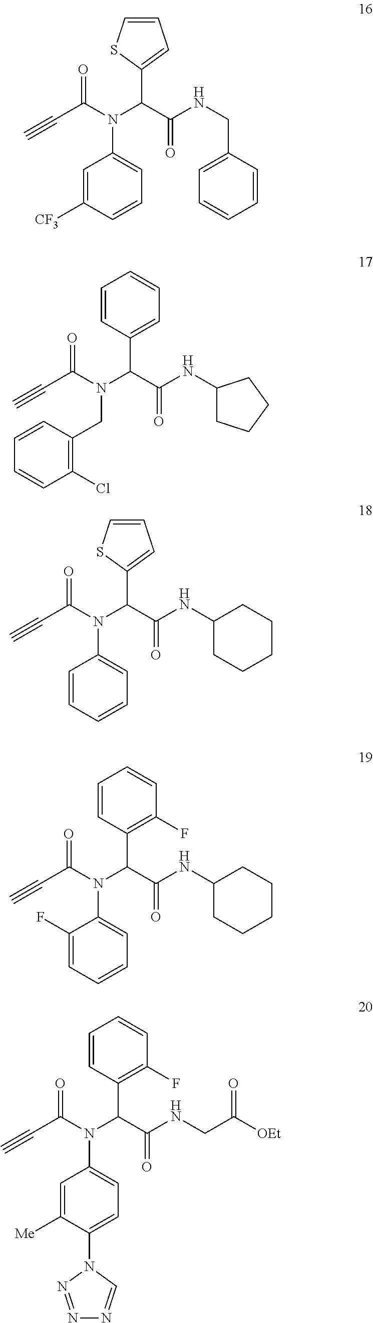

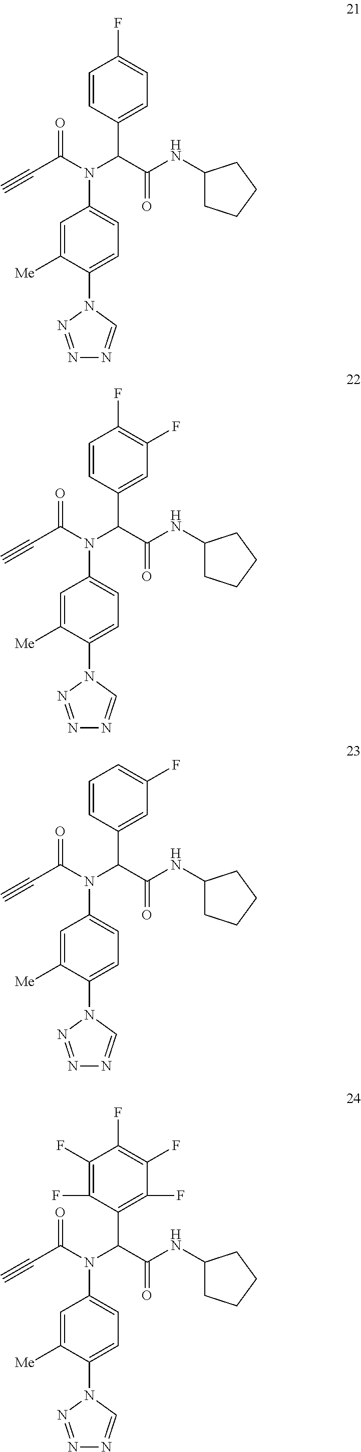

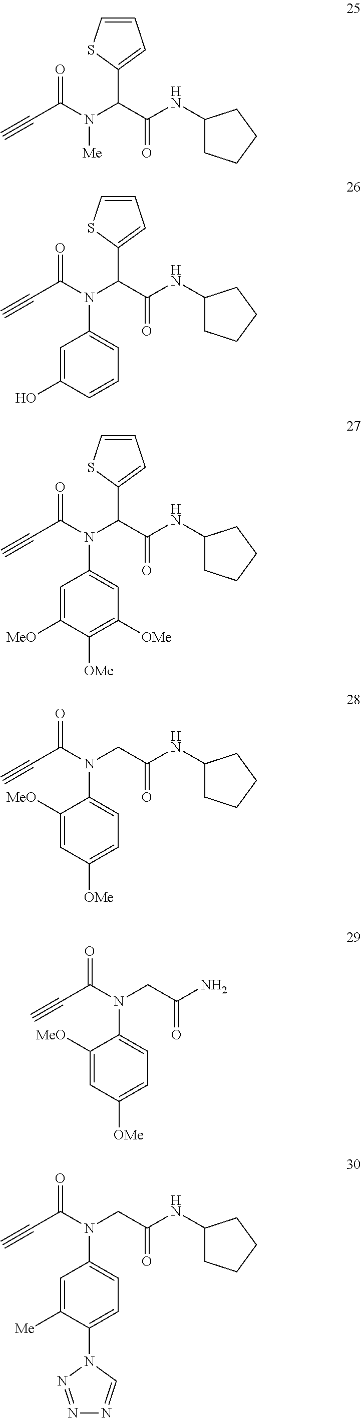

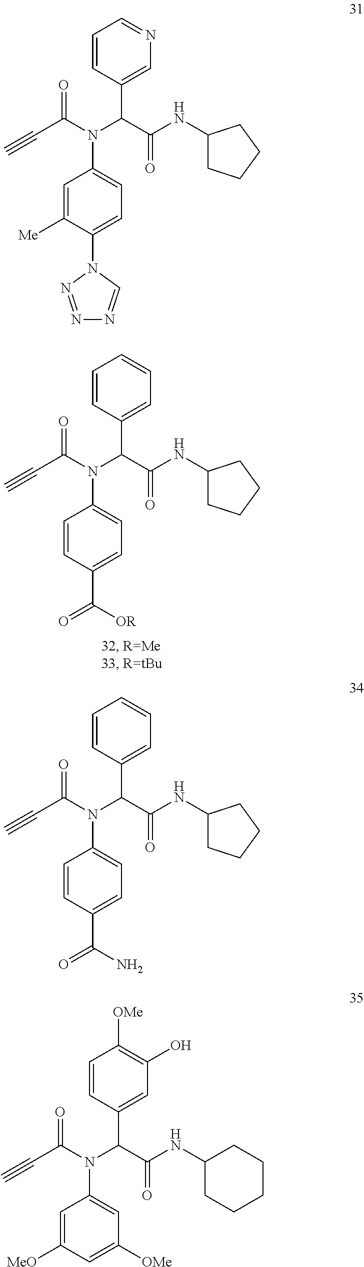

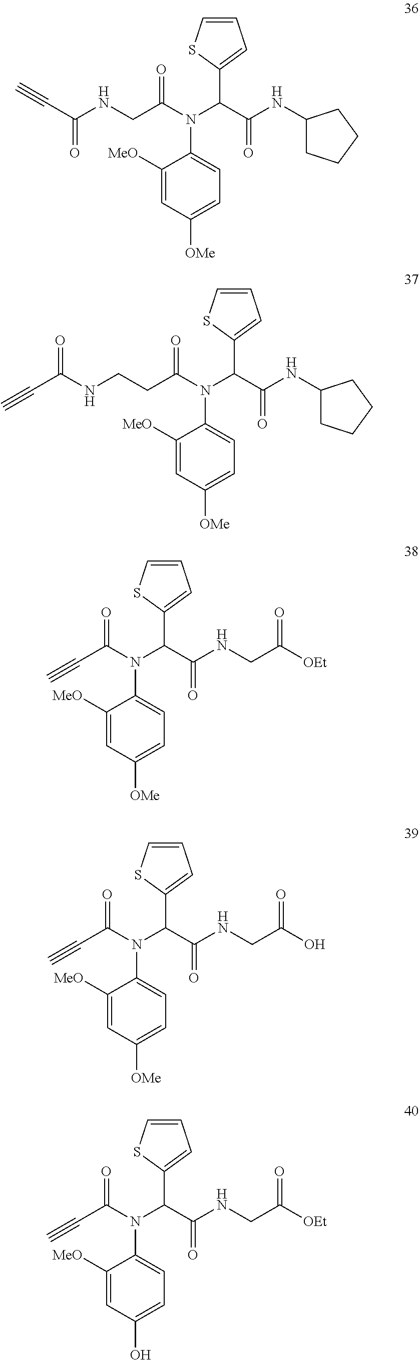

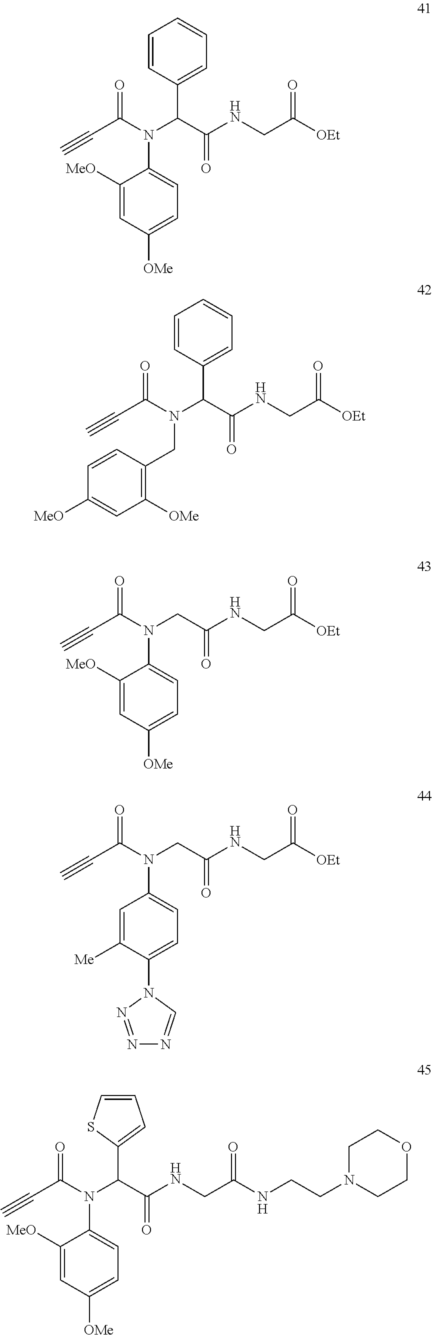 [SMILES: C#CC(=O)N(c1ccc(OC)cc1OC)C(C(=O)NCC(=O)OCC)c1ccccc1 ; C#CC(=O)N(Cc1ccc(OC)cc1OC)C(C(=O)NCC(=O)OCC)c1ccccc1 ; C#CC(=O)N(CC(=O)NCC(=O)OCC)c1ccc(OC)cc1OC ; C#CC(=O)N(CC(=O)NCC(=O)OCC)c1ccc(-n2cnnn2)c(C)c1 ; C#CC(=O)N(c1ccc(OC)cc1OC)C(C(=O)NCC(=O)NCCN1CCOCC1)c1cccs1]

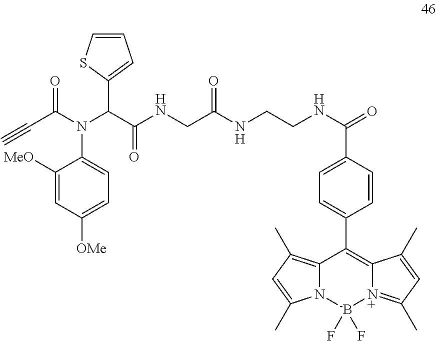

7. A pharmaceutical composition comprising any of the compounds of claims 1 to 3 and a pharmaceutically acceptable carrier.

8. A method for treatment a cancer subject, comprising the step of:
administering to a person in need of said treatment method an effective amount of the pharmaceutical composition according to claim 4.

9. A method of claim 8, where the cancer is: breast cancer, ovarian cancer, prostate cancer, colon cancer, brain cancer, pancreatic cancer, skin cancer, lung cancer, and multiple myeloma.

10. A method for promoting the release and nuclear localization of the transcription factor Nrf2 comprising the step of:
administering an effective amount of the pharmaceutical compositions according to claim 4.

10. A biological or medical diagnostic imaging technique, comprising:
administering to a subject a compound of claims 4 or 5 to a subject to be imaged; and
detecting a signal from said compound using a detector for the imaging technique.
